SEQUENCE LISTING (1 sequence)

SEQ ID NO: 1 - DNA, 21 bp, Artificial Sequence
  Description of Artificial Sequence Synthetic primer
catgcaagcttcagggttgag

(19) United States
(12) Patent Application Publication
(10) Pub. No.: US 2011/0135689 A1
(43) Pub. Date: Jun. 9, 2011

(54) Vaccine, therapeutic composition and methods for treating or inhibiting francisella tularenis

(75) Inventors: Michael A. Apicella, Iowa City, IA (US); Molly K. McLendon, Iowa City, IA (US); Bradford W. Gibson, Novato, CA (US); Deborah M.B. Post, Novato, CA (US)

(73) Assignees: University of Iowa Research Foundation, Iowa City, IA (US); Buck Institute for Age Research, Novato, CA (US)

(21) Appl. No.: 12/927,912

(22) Filed: Nov. 29, 2010

Related U.S. Application Data
(60) Provisional application No. 61/283,170, filed on Nov. 30, 2009.
(60) Provisional application No. 61/348,515, filed on May 26, 2010.

(51) Int. Cl. A61K 39/02; C12N 5/00; C07K 16/12; C07K 14/00; C07H 21/00; C07H 1/00

(52) U.S. Cl. 424/234.1; 435/325; 530/387.1; 530/387.3; 530/395; 536/22.1; 536/123.1

(57) ABSTRACT

The present invention is directed to compounds and methods for treating a mammal exposed to Francisella tularensis.

### RELATED APPLICATIONS

This application claims priority to U.S. Provisional Patent Application No. 61/283,170 filed Nov. 30, 2009 and U.S. Provisional Patent Application No. 61/348,515 filed May 26, 2010, the entirety of which are incorporated herein by reference.

### U.S. GOVERNMENT RIGHTS

This work was supported by the National Institutes of Allergy and Infectious Diseases Grant P01AI44642 and R01 AI063441, and Public Health Service grant P01AI4464 and, AI057160. The United States Government has certain rights to this invention.

### BACKGROUND OF THE INVENTION

The bacteria Francisella tularensis has four identified subspecies—Francisella tularensis (type A), Francisella holarctica (type B), Francisella mediasiatica and Francisella novicida. The type that is historically associated with human infections in the U.S. is Francisella tunarensis tularensis (type A), and generally, this bacterium was associated with infection of rural populations. It is a hardy organism that can survive low temperatures, in water, moist soil, hay, straw or decaying animal carcasses. Naturally occurring infections by this bacteria in the U.S. have become more and more rare over the past century, reaching <200 cases/yr in the 1990s. The general course of fatal infection is respiratory failure, shock and death. The ulceroglandular form of disease is usually caused by percutaneous inoculation with the organism and causes ulceration of the skin and lymph node swelling. F. tularensis is typically transferred to humans through bites from infected arthropods (usually ticks), contact with infected animal tissues or fluids, direct contact with or ingestion of contaminated water, food or soil or inhalation of aerosolized bacteria. No person-to-person transfer has been documented. It is associated with a 5% mortality rate. The pneumonic form of infection is caused when the organism is aerosolized. As few as 10-50 organisms can infect humans. The mortality by this pneumonic route of infection is 30% with 70% morbidity. The mechanism of virulence for F. tunarensis is not well understood.

F. tularensis has been the target of several vaccines over the years (killed cell, live attenuated and subunit). The U.S., a small number of test subjects have been inoculated with a killed cell vaccine. More recently, a F. tularensis Live Vaccine Stain (LVS), an attenuated live cell vaccine (holarctica) that is effective against low dose infections, has been tested. LVS was modified from a live cell vaccine that had been used extensively in Russia (mostly European type B). Neither of these vaccines is available to the public.

### SUMMARY OF THE INVENTION

The present invention provides a composition comprising purified Francisella tularensis Polysaccharide Capsule (PC). PC is sometimes referred to as “Capsular Antigen” (CA). PC is a complex carbohydrate containing a tetrasaccharide repeat of one residue of 2-acetamido-2,6-dideoxy-o-glucose (o-QuiNAc), one residue of 4,6-dideoxy-4-formamido-D-glucose (o-Qui4NFm), and two residues of 2-acetamido-2-deoxy-o-galacturonamide (o-GalNAcAN). Each tetrasaccharide unit is about 792 daltons in size, and in certain embodiments, the PC has between about 250 to 500 tetrasaccharide units. The order of the residues appears to be the following:

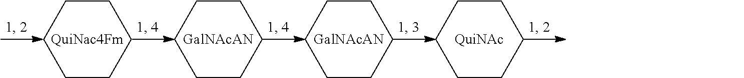

PC from Francisella tularensis is distinguishable from lipopolysaccharide (LPS) from Francisella tularensis, even though both molecules are formed from the same saccharide building blocks. In certain embodiments, the PC has a molecular weight of 200 kDa to 400 kDa. In contrast, the molecular weight of LPS is about 65 kDa. In certain embodiments, the composition contains between 0% and 10% by weight of LPS from Francisella tularensis. In certain embodiments, the composition contains less than 1% by weight of LPS from Francisella tularensis. In certain embodiments, the composition contains less than 0.1% by weight of LPS from Francisella tularensis. In certain embodiments, the composition contains less than 1% by weight LPS core sugars KDO or mannose. In certain embodiments, the composition contains less than 0.1% by weight LPS core sugars KDO or mannose. In certain embodiments, the composition contains less than 1% by weight of lipid A. In certain embodiments, the composition contains less than 0.1% by weight of lipid A.

In certain embodiments, the composition further comprises a physiologically-acceptable, non-toxic vehicle. In certain embodiments, the composition further comprises an adjuvant.

The present invention provides complex comprising the composition as described above operably linked to a conjugation molecule. In certain embodiments, the conjugation molecule is a peptide, a nucleic acid, or a polysaccharide that is not PC. In certain embodiments, the PC is conjugated to tetanus toxoid.

The present invention provides a method of eliciting an immune response in an animal comprising introducing into the animal the composition or complex described above.

The present invention provides a method of generating antibodies specific for PC, comprising introducing into the animal the composition or complex described above. In certain embodiments, the method further comprises introducing a second dose of the composition or complex into the animal. In certain embodiments, the composition or complex is introduced intranasally or dermally.

The present invention provides a vaccine comprising an immunogenic amount of purified Francisella tularensis PC, which amount is effective to inhibit in a patient infection by Francisella tularensis, in combination with a physiologically-acceptable, non-toxic vehicle.

The present invention provides a purified antibody that binds specifically to PC from Francisella tularensis. As used herein, the term “antibody” includes scFv, humanized, fully human or chimeric antibodies, single-chain antibodies, diabodies, and antigen-binding fragments of antibodies (e.g., Fab fragments).

The present invention provides a composition comprising a purified antibody that binds specifically to PC from Francisella tularensis and a pharmaceutically acceptable carrier. In certain embodiments, the antibody is a human antibody or a humanized antibody. A “humanized” antibody contains only the three CDRs (complementarity determining regions) and sometime a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes. In certain embodiments, the antibody binds to lipopolysaccharide (LPS) from Francisella tularensis at less than 1% as compared to binding to PC. In certain embodiments, the antibody is a single-chain Fv or an scFv fragment. An scFv fragment is where a light chain variable region of a monoclonal antibody is recombinantly fused through a linker sequence to a heavy chain variable region of the antibody. In certain embodiments, the PC comprises an epitope with the structure that is distinct from the O-antigen, as monoclonal antibodies specific for PC do not bind to the O-antigen and mice immunized with PC do not develop antibodies to the O-antigen.

The present invention provides a monoclonal antibody having the same epitope specificity as hybridoma 11B7, deposited with the American Type Culture Collection (ATCC®, 10801 University Boulevard, Manassas, Va. 20110-2209 USA) on Jan. 20, 2010, and given Patent Deposit Designation PTA-10595. The deposit will be maintained in the ATCC® Depository, which is a public depository, for a period of 30 years, or 5 years after the most recent request, or for the enforceable life of the patent, whichever is longer, and will be replaced if it becomes nonviable during that period. In certain embodiments, the monoclonal antibody is produced by hybridoma 11B7, ATCC® Patent Deposit Designation PTA-10595. In certain embodiments, the antibody is operably linked to an imaging agent. Imaging agents of the present invention include fluorescent, radioactive, and enzymatic labels, among other things.

The present invention provides a composition comprising an antibody as described above and a pharmaceutically acceptable carrier.

The present invention provides a cell of hybridoma 11B7, ATCC® Patent Deposit Designation PTA-10595.

The present invention provides a method of making an antibody, comprising immunizing a non-human animal with an immunogenic amount of PC.

The present invention provides a method of making an antibody, comprising providing a hybridoma cell that produces a monoclonal antibody specific for PC, and culturing the cell under conditions that permit production of the monoclonal antibody.

The present invention provides a method of inhibiting Francisella tularensis infection in a patient, comprising administering to the patient a composition comprising the antibody as described above, wherein the antibody is a humanized antibody. In certain embodiments, the antibody is administered intravenously or intramuscularly.

The present invention provides a method of determining whether a biological sample contains Francisella tularensis bacteria, comprising contacting the sample with the antibody as described above and determining whether the antibody specifically binds to the sample, wherein the biding is an indication that the sample contains a Francisella tularensis bacteria.

The present invention provides a method of treating a Francisella tularensis infection, comprising administering to a patient the antibody as described above.

The present invention provides a method of purifying PC from a biological sample containing PC, comprising providing an affinity matrix comprising an anti-PC antibody as described above bound to a solid support; contacting the biological sample with the affinity matrix to produce an affinity matrix-PC complex; separating the affinity matrix-PC complex from the remainder of the biological sample; and releasing PC from the affinity matrix.

The present invention provides a method of diagnosing Francisella tularensis infection in a patient comprising providing a biological sample from a mammal suspected of having a Francisella tularensis infection, contacting the sample with the antibody as described above to form a sample/antibody complex, and determining the presence of the sample/antibody complex, wherein the presence of the sample/antibody complex is indicative of infection. In certain embodiments, the mammal is a human. In certain embodiments, the biological sample comprises blood.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1. FIG. 1 shows a Western blot of six F. tularensis strains using MAb 11B7. This antibody binds to capsule from lysates of five F. tularensis strains from the Oregon State Health lab (lanes 1-5) and F. tularensis subsp holarctica 1547 (lane 6).

FIGS. 2A-2D. FIG. 2 shows the results of immunoelectron microscopic studies of surface labeling with MAb 11B7. FIG. 2A is a whole mount immunoelectron microscopy study that demonstrates surface labeling of F. tularensis SCHU S4 with 12 nm colloidal gold particles and FIG. 2B is a secondary only control for the whole mount study. FIG. 2C shows a cryo-immunoelectron micrograph of F. tularensis LVS stained with MAb 11B7 and 12 nm colloidal gold. It shows circumferential staining of capsule material on the surface of the organism. FIG. 2D shows the secondary antibody control section. Magnification is 15,000.

FIGS. 3A and 3B. FIGS. 3A and 3B demonstrate chromatographic separation using Sephacryl SH500 that shows the separation of Francisella capsule from the LPS. FIG. 3A is a ProQ Emerald green stained SDS-PAGE showing the results of column separation. The capsule can be seen in lanes 13 and 14 and the LPS in lanes 21 and 23. Lane S is an aliquot of the sample after proteinase K and phenol extraction and prior to Triton X-114 extraction. FIG. 3B is an immunodot assay of each fraction using MAb 11B7. The numbers in the panels correspond to aliquots from each one ml fraction from the column.

FIGS. 4A-4D. FIG. 4A shows a silver stained gel, FIG. 4B shows a ProQ Emerald Green glycostained gel, FIG. 4C shows a Western blot with the anti-capsule MAb 11B7 and FIG. 4D shows a Western blot with MAb anti-LPS MAb FB11. Lane S is the molecular weight standard, lane 1 contains the crude capsule prep before Triton X114 treatment, lane 2 contains the capsule preparation after Triton X-114 treatment and lane 3 contains LPS separated into the Triton X114 fraction. This contains minimal amounts of residual capsule.

FIGS. 5A-5D. FIGS. 5A-5D show positive ion vMALDI-LIT mass spectra of unprocessed capsule. Gas-phase fragmentation of the intact capsule allowed fragments of the capsule to be observed by MS analyses. The MSⁿ spectrum (FIG. 5A) of the capsule demonstrated that the predominant mass observed in the spectrum was at m/z 793. The presence of structures consisting of a 792 Da saccharide repeating unit with one (m/z 793), two (m/z 1585), three (m/z 2377), or four (m/z 3170) saccharide repeats were detected. MSⁿ analyses, using collisionally induced dissociation, of a single unit tetrasaccharide at 793 m/z (FIG. 5B) yielded a number of fragment ions. The predominant fragment ions were sequential fragmentated to yield MS³ data of m/z 620 (FIG. 5C) and MS⁴ data of m/z 404 (FIG. 5D). The MS⁴ data of m/z 404 demonstrated that it is composed of two carbohydrate monomers with nominal masses of 187 and 216 Da, and that these sugars are located adjacent to one another. Masses labeled with an * designate major masses +/− water.

FIGS. 6A-6C. Negative-ion vMALDI-MS analyses of LPS (FIG. 6A) and capsule without (FIG. 6B) and with (FIG. 6C) HF treatment. The F. tularensis tetraacyl lipid A is readily detected in the LPS sample, but is not seen in either of the capsule samples.

FIG. 7. FIG. 7 demonstrates ¹H-¹³C HMQC spectrum of F. tularensis capsule polysaccharide collected at 600 MHz and plotted near the noise floor. Methyl and formyl resonances were aliased in the ¹³C dimension to optimize the spectral window. Assignments for each sugar in the core tetrasaccharide are indicated in a different color, and the dashed lines connect the resonances within each spin system. The indicated assignments are highly consistent with those for the O-antigen tetrasaccharide (Conlan J W, Shen H, Webb A, Perry M B (2002) Mice vaccinated with the O-antigen of Francisella tularensis LVS lipopolysaccharide conjugated to bovine serum albumin develop varying degrees of protective immunity against systemic or aerosol challenge with virulent type A and type B strains of the pathogen. Vaccine 20: 3465-3471; Thirumalapura N R, Goad D W, Mort A, Morton R J, Clarke J, et al. (2005) Structural analysis of the O-antigen of Francisella tularensis subspecies tularensis strain OSU 10. J Med Microbiol 54: 693-695).

FIG. 8. FIG. 8 is a Western blot developed with MAb 11B7 showing whole organism lysates from F. tularensis 1547 (lane 1), F. tularensis LVS wbtI, wbtA2, wbtA1, capB, capC, and wbtC in lanes 2-7 respectively. F. tularensis LVS is in lane 8.

FIGS. 9A-9B. FIGS. 9A and 9B are Western blots which demonstrates loss of O-antigens but persistence of capsule in F. tularensis lpxL. FIGS. 21A and 21B show F. tularensis LVS (lane 1), F. tularensis 1547lpxL (lane 2) and F. tularensis 1547lpxL complemented with lpxL (lane 3). FIG. 9A was reacted with the F. tularensis anti-LPS MAb FB11 and FIG. 9B was reacted with the F. tularensis anti-capsule MAb 11B7.

FIG. 10. FIG. 10 shows the results of protection of BALB/c mice passively immunized with MAb 11B7 from lethal infection due to F. tularensis LVS. Eight mice received 75 μg of MAb 11B7 and were challenged with 10⁴ cfu (□), eight mice received 75 μg of MAb 11B7 and were challenged with 10⁵ cfu (Δ) and eight mice received 75 μg of MAb 2C3 a Neisseria H.8 MAb as a negative control (♦) and were challenged with 10⁴ cfu. As can be seen, MAb 11B7 was protective while all of the mice receiving the control antibody died by day 6 after challenge.

FIG. 11. FIG. 11 shows the results of immunization of BALB/c mice with 2 doses of 10 μg F. tularensis capsular antigen at a four week interval. Capsule immunized mice were challenged a week after the second dose with either 10⁴ (X) or 10⁵ () F. tularensis LVS. Control mice received PBS and were challenged with either 250 cfu (Δ) or 750 cfu (▪) of F. tularensis LVS. As can be seen, these results indicate that the capsular antigen is protective against a challenge at least 40-fold higher than the LD₅₀ for F. tularensis in mice. Each group contains 5 mice.

FIG. 12A-12C. FIG. 12A-12B is a composite Western blot using sera from capsule immunized mice. A goat anti-mouse IgG peroxidase conjugate was used to develop panel A and a goat anti-mouse IgM peroxidase conjugate was used in panel B. All sera were tested at a 1:1000 dilution. C1 and C2 represent sera from animals given PBS only. One strip has been reacted with MAb 11B7 as a control for capsule using the goat anti-mouse IgG peroxidase conjugate.

FIG. 12C shows a Western Blot that demonstrates that capsule does not appear to be shed during growth into the supernate of liquid cultures. One microgram of each of the following underwent SDS-PAGE using a 4-12% gel followed by transfer to nitrocellulose. Lane 1 contains the molecule weight controls and the arrow indicates 200 kDa, lane 2 is an organism pellet loaded into sample buffer, lane 3 is purified LVS capsule, lane 4, 5 and 6 are 70% ethanol precipitates of 4 (lane 5), 8 (lane 6) and 24 (lane 7) hour supernates cleared of organisms by centrifugation at 13,500 g. The bacterial pellets removed from the 4, 8 and 24 hour supernates are in lanes 8, 9 and 10 respectively. Lane 4 and 11 contained no sample. Lane 12 contains an TX-114 extracted LPS sample. The Western blot was developed with MAb 11B7 at a dilution of 1:10,000. These studies show that the majority of the LVS capsule is associated with the organism and only minimal amounts of capsule are released into the broth supernate at 24 hours.

FIG. 13. Positive-ion MALDI_TOF mass spectra of unprocessed capsule. The predominant monoisotopic mass observed is m/z 815, this mss corresponds to the sodiated form of the 792 Da tetrasaccharide repeating unit. These data show that we were able to detect up to six repeating units in the capsule sample.

FIG. 14. Positive-ion vMALDI-LIT mass spectra of HF-treated capsule. After HF treatment the predominant sodiated monoisotopic masses observed are at m/z 833 and 1625 which corresponds to one or two units of the 792 Da tetrasaccharide repeat, respectively. The fragments observed after HF treatment were generated by chemical hydrolysis and therefore contain an additional water (793+18=811 Da) relative to the peaks observed in the unprocessed capsule sample that were generated by gas-phase fragmentation. Masses labeled with an * designate major masses minus water.

FIGS. 15A-15D. Positive in-vMALDI-MSⁿ analysis of unprocessed capsule. The tetrasaccharide at m/z 793 (A) was sequentially fragmented to yield fragment ions at m/z 606 (B), at m/z 433 (C), and at m/z 390 (D). The MS⁴ data of m/z 433 demonstrated that it is composed of two carbohydrate monomers of the same mass, 216 Da, that are adjacent to one another. The MS⁴ data of m/z 390 demonstrated that it is composed of two carbohydrate monomers with masses of 173 Da and 216 Da that are located adjacent to on another. Masses labeled with an * designate major masses +/− water.

FIGS. 16A-16C. Positive in-vMALDI-MSⁿ analyses of unprocessed capsule. The tetrasaccharide at m/z 793 (A) was sequentially fragmented to yield fragment ions at m/z 577 (B), and at m/z 361 (C). The MS⁴ data of m/z 361 demonstrated that it is composed of two carbohydrate monomers of masses of 173 Da and 187 Da that are adjacent to one another. Masses labeled with an * designate major masses +/− water.

FIGS. 17A-17C. (A) Total ion chromatogram of alditol acetate (AA) derivatives of F. tularensis capsule. Three major peaks were observed. One of these peaks could be assigned to QuiNAc (RT 23.56 min), based on C1 (B) and E1 (C) data. The two mass fragments observed in E1 mode at m/z 302 and 144 C1 are consistent with the QuiNAc assignment. E1 analyses suggested that the unknown peaks are most likely anhydro-degredation products of QuiNAc or de-formylated Qui4NFm. No peak was observed that was consistent with HexNAcAN; it has been previously suggested that this sugar is either too labile or polar to be observed by these analyses (Y. A. Knirel, et al. Eur. J. Biochem. 1985. 150:541-550). Inositol was spiked in as a control.

FIGS. 18A-18C. (A) Total ion chromatogram of alditol acetate (AA) derivatives of F. tularensis LPS. QuiNAc, GlcNAc, GalNAc, and two unknown peaks were detected in these samples. C1 (B) and E1 (C) analyses verified the presence of QuiNAc. The two mass fragments observed in E1 mode at m/z 302 and 144 C1 are consistent with the QuiNAc assignment. C1 and E1 analyses also suggested that the unknown peaks are most likely ahydro degradation production of QuiNAc or de-formylated Qui4NFm. Inositol was spiked in as a control.

FIGS. 19A-19C. Total ion chromatogram of trimethylsilyl (TMS) derivative of the capsule sample. QuiNAc and Hex NAcAN were detected in these samples. Fragmentation analysis verified their identification (B and C, respectively). Mannitol was included as a control.

FIGS. 20A-20B. (A) Total ion chromatogram of trimethylsilyl (TMS) derivative of F. tularensis LPS scanned over a mass range of m/z 50-600. These data show that the main constituents observed in this sample include lipid A components as well as core sugars. (B) Selected ion chromatogram of the LPS sample, to scan for amino sugars. The sample was scanned over the mass range of m/z 172.5-173.5. These data confirmed the presence of QuiNAc, GalNAc, and GlcNAc.

FIGS. 21A-21C. Total ion chromatogram of partially methylated alditol acetate (PMAA) derivatives of F. tularensis capsule. Data was collected over a mass range of m/z 50-600. QuiNAc with a 3-linkage and a terminal linkage were identified at 25.40 and 22.57 min, respectively. Fragmentation analysis verified these assignments (B and C, respectively).

### DETAILED DESCRIPTION OF THE INVENTION

### I. Polysaccharide Capsule (PC)

The present technology is a newly discovered component of the outer surface of F. tularensis bacteria, the polysaccharide capsule (PC) or “Capsular Antigen” (CA). The building blocks of PC are chemically identical to the O-antigen of LPS, but PC is distinguishable from the O-antigen of LPS in that it is much larger, and is linked to a different lipid host. This antigen has been used to develop a monoclonal antibody (clone 11B7) that is specific against it, which can be utilized for passive immunity in acute infections. The 11B7 antibody specifically recognizes Ft PC, and does not bind to Ft LPS.

PC carbohydrate can be prepared as follows. Francisella tularensis ssp tularensis is grown on Chamberlin's media for 48 hours. The organisms are harvested from the plates into 100 mM NaCl, 15 mM Tris pH 7.4 containing 2% SDS. The organisms are allowed to lyse for one hour at 70° C. in a water bath. Proteinase K (100 mcg/ml) is added and the solution allowed to incubate at 37° C. overnight. One-tenth of the volume of sodium acetate is added and the PC is precipitated by the addition of 3 volumes of absolute ethanol. The precipitate is separated by centrifugation at 3000×g and the supernatant is removed. The precipitate is solubilized in 0.3M sodium acetate and re-precipitated in 3 volumes of ethanol. This is repeated three times to remove residual SDS. After the final precipitation, the precipitate is raised in 10 mM Tris pH 7.0 and 10 mcg/100 ml of staphylococcal nuclease is added to digest any nucleic acids. This is incubated overnight at 37° C. After incubation, an equal volume of 95% phenol is added to the PC solution. This is heated to 65° C. for 1 hour, cooled to 4° C. and centrifuged at 3000×g for 15 minutes at 4° C. The aqueous phase is removed and saved. The phenol phase is re-extracted once with distilled water and the above process repeated. The aqueous phase is added to the original and to this is added enough 3M sodium acetate to raise the final concentration to 300 mM sodium acetate. This solution is precipitated with 2 volumes of absolute ethanol and the precipitate is collected by centrifugation. This is raised in 0.3M sodium acetate and re-precipitated with ethanol. This is repeated three times to remove any residual phenol. After the final precipitation, the PC is raised in distilled water and extracted with enough triton X-114 to raise the concentration to 5%. This is vortexed and placed at 4° C. overnight and then at 37° C. for 4 hours. The solution is centrifuged at 3000×g for 15 minutes at 37° C. and the aqueous phase recovered. This is solution is dialyzed against distilled water×3 changes over 48 hours. The final solution is lyophilized and is the purified PC.

### II. Complexes of PC and Other Molecules

In certain embodiments, the PC can be conjugated or linked to a peptide or to another polysaccharide. In certain embodiments, PC is conjugated to tetanus toxoid. In certain embodiments, PC is conjugated to meningococcal porin.

For example, immunogenic proteins well-known in the art, also known as “carriers,” may be employed. Useful immunogenic proteins include keyhole limpet hemocyanin (KLH), bovine serum albumin (BSA), ovalbumin, human serum albumin, human gamma globulin, chicken immunoglobulin G and bovine gamma globulin. Useful immunogenic polysaccharides include group A Streptococci polysaccharide, C-polysaccharide from group B Streptococci, or the capsular polysaccharide of Streptococci pnuemoniae. Alternatively, polysaccharides of other pathogens that are used as vaccines can be conjugated or linked to the PC.

### III. Vaccines of the Invention

The present invention provides a vaccine for use to protect mammals against the colonization and/or infection of F. tularensis bacteria. In one embodiment of this invention, PC can be delivered to a mammal in a pharmacologically acceptable vehicle. As one skilled in the art will appreciate, it is not necessary to use the entire PC complex carbohydrate. A selected portion of the PC complex carbohydrate, for example the epitope that specifically binds to antibody 11B7, can be used.

As one skilled in the art will also appreciate, it is not necessary to use a PC that is identical to native PC. The modified PC can correspond essentially to the corresponding native PC. As used herein “correspond essentially to” refers to a PC epitope that will elicit a immunological response at least substantially equivalent to the response generated by a native PC. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest. Vaccines of the present invention can also include effective amounts of immunological adjuvants, known to enhance an immune response.

To immunize a subject, the PC, or an immunologically active fragment, variant or mutant thereof, is administered parenterally, usually by intramuscular or subcutaneous injection in an appropriate vehicle. Other modes of administration, however, such as oral, intranasal or intradermal delivery, are also acceptable.

Vaccine formulations will contain an effective amount of the active ingredient in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the biofilm peptide or fragment thereof in one or more doses. Multiple doses may be administered as is required to maintain a state of immunity to the bacterium of interest, e.g., F. tularensis.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

To prepare a vaccine, the purified PC, fragment, or variant thereof, can be isolated, lyophilized and stabilized, as described above. The PC may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine formulation, or may be conjugated to proteins such as keyhole limpet hemocyanin (KLH) or human serum albumin (HSA) or other polymers.

The application of PC or variant thereof, for vaccination of a mammal against colonization of F. tularensis offers advantages over other vaccine candidates.

### IV. PC Antibodies and Methods of Making Anti-PC Antibodies

The present inventors cloned hybridomas that produced monoclonal antibodies against polysaccharide capsule (PC). The inventors screened the hybridomas for antibodies binding to PC, and succeeded in isolating an anti-PC antibody (11B7) that have a high affinity specifically for PC but not for LPS. The anti-PC antibodies were capable of passively protecting a mammal from challenge with F. tularensis.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, more preferably at least 75%, even more preferably at least 80%, still more preferably at least 85%, yet more preferably at least 90%, and most preferably at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

Polyclonal and monoclonal antibodies can be prepared by methods known to those skilled in the art. For example, the antibodies can be prepared by the methods described below.

PC to be used for the immunization of animals includes the full-length PC, comprising its entire complex carbohydrate structure, or to the epitope that induces the production of 11B7 antibodies. As the antigen for immunization, PC itself, or its structure, can be used without modification, or after being conjugated with a carrier molecule. When a carrier molecule is used, for example, the antigen PC is first coupled with the carrier molecule, and then an adjuvant is added thereto. Such adjuvants include Alum, Freund's complete and incomplete adjuvants and the like, any of which can be combined together.

An antigen prepared as described above is given to a mammal, such as a mouse, rat, hamster, guinea pig, horse, monkey, rabbit, goat, and sheep. This immunization can be performed by any existing method, including typically used intravenous injections, subcutaneous injections, and intraperitoneal injections. There are no restrictions as to the immunization intervals. Immunization may be carried out at intervals of several days to several weeks, preferably four to 21 days. A mouse can be immunized, for example, at a single dose of 10 to 100 μg (for example, 20 to 40 μg) of the antigen protein, but the dose is not limited to these values.

Before the first immunization, and three to seven days after the second and subsequent immunizations, blood is collected from the animals, and the sera are analyzed for antibody titer. To promote an immune response, an aggregating agent such as alum is preferably used. In general, selected mammalian antibodies have sufficiently high antigen binding affinity. Antibody affinity can be determined using a saturation binding assay, an enzyme-linked immunosorbent assay (ELISA), or a competitive assay (for example, radioimmunoassay).

Polyclonal antibodies can be screened by a conventional crosslinking analysis, such as that described in “Antibodies, A Laboratory Manual (Cold Spring Harbor Laboratories, Harlow and David Lane edit. (1988)).” An alternative method is, for example, epitope mapping (Champe et al., J. Biol. Chem. 270:1388-1394 (1995)). A preferred method for determining polypeptide or antibody titers comprises quantifying antibody-binding affinity. In other embodiments, methods for assessing one or more biological properties of an antibody are also used in addition to or instead of the methods for determining antibody-binding affinity. Such analytical methods are particularly useful because they demonstrate the therapeutic effectiveness of antibodies. When an antibody exhibits an improved property in such analysis, its binding affinity is generally, but not always, enhanced.

Hybridomas that are used to prepare monoclonal antibodies can be obtained, for example, by the method of Milstein et al. (Kohler, G., and Milstein, C., Methods Enzymol. 1981, 73, 3-46). Myeloma cells to be fused with antibody-producing cells may be cell lines derived from any of the various animals, such as mice, rats, and humans, which are generally available to those skilled in the art. The cell lines to be used are drug-resistant, and cannot survive in a selective medium (e.g., HAT medium) in an unfused state, but can survive in a fused state. 8-azaguanine-resistant cell lines are generally used, which are deficient in hypoxanthine-guanine-phosphoribosyl transferase and cannot grow in a hypoxanthine-aminopterin-thymidine (HAT) medium. Myeloma cells include a variety of known cell lines, for example, P3x63Ag8.653 (J. Immunol. (1979) 123: 1548-1550), P3x63Ag8U.1 (Current Topics in Microbiology and Immunology (1978) 81: 1-7), NS-1 (Kohler, G. and Milstein, C., Eur. J. Immunol. (1976) 6: 511-519), MPC-11 (Margulies, D. H. et al., Cell (1976) 6: 405-415), SP2/0 (Shulman, M. et al., Nature (1978) 276: 269-270), F0 (de St. Groth, S. F. et al., J. Immunol. Methods (1980) 35: 1-21), S194 (Trowbridge, I. S., J. Exp. Med. (1978) 148: 313-323), R210 (Galfre, G. et al., Nature (1979) 277: 131-133), and P3U1 (J. Exp. Med. 1979, 150:580; Curr Top Microbiol. Immunol. 1978, 81:1). Human myeloma and mouse-human heteromycloma cell lines can also be used to produce human monoclonal antibodies (Kozbar, J. Immunol. 133:3001 (1984); Brodeur et al., Monoclonal Antibody Production Techniques and Application, pp. 51-63 (Marcel Dekker, Inc., New York, 1987)). Antibody-producing cells are collected, for example, from animals sacrificed two to three days after the final immunization. Antibody-producing cells include spleen cells, lymph node cells, and peripheral blood cells. Spleen cells are generally used. Specifically, tissues such as spleens or lymph nodes are excised or collected from the various animals described above. Then, the tissues are crushed and the resulting material is suspended in a medium or buffer, such as PBS, DMEM, or RPMI1640, followed by filtration with a stainless mesh or the like. This is then centrifuged to obtain antibody-producing cells of interest.

The above-described myeloma cells and antibody-producing cells are then fused. Cell fusion is achieved by contacting the myeloma cells with the antibody-producing cells at a ratio of 1:1 to 1:20 in a medium for animal cell culture, such as MEM, DMEM, and RPMI-1640, at 30 to 37° C. for one to 15 minutes in the presence of a fusion-promoting agent. To promote cell fusion, the antibody-producing cells and the myeloma cells may be fused using a commercially available cell-fusion device, using a fusion-promoting agent, such as polyethylene glycol (mean molecular weight 1,000 to 6,000 (Da)) or polyvinyl alcohol, or a virus for fusion, such as Sendai virus.

Hybridomas of interest are selected from the cells after cell fusion. The selection methods include methods using selective propagation of cells in a selective medium. Specifically, a cell suspension is diluted with an appropriate medium, and then the cells are plated on to microtiter plates. An aliquot of selection medium (for example, HAT medium) is added to each well, and then the cells are cultured while the selection medium is appropriately exchanged. The cells grown as a result can be saved as hybridomas.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

The antibodies of the present invention are antibodies that provide passive immunity to an F. tularensis infection.

Methods for preparing monoclonal antibodies from the obtained hybridomas include standard cell culture methods and methods comprising ascites production. In cell culture methods, hybridomas are cultured for two to 14 days under standard culture conditions (for example, at 37° C. at 5% CO₂ atmosphere), in a culture medium for animal cells, such as RPMI-1640 or MEM containing 10 to 20% fetal calf serum, or serum-free medium, and antibodies are then prepared from the culture supernatant. In the method comprising ascites production, hybridomas are administered to the peritoneal cavities of mammalian individuals of the same species as that from which the myeloma cells are derived, and the hybridomas proliferate in to large quantities. Ascites or serum is then collected after one to four weeks. To enhance ascites production, for example, pristane (2,6,10,14-tetramethylpentadecane) may be pre-administered to the peritoneal cavity.

Antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant antibodies, produced by gene engineering. The genes encoding the antibodies obtained by a method described above are isolated from the hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The present invention provides the nucleic acids encoding the antibodies of the present invention, and vectors comprising these nucleic acids. Specifically, using a reverse transcriptase, cDNAs encoding the variable regions (V regions) of the antibodies are synthesized from the mRNAs of hybridomas. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. The present invention provides cells expressing antibodies of the present invention. The cells expressing antibodies of the present invention include cells and hybridomas transformed with a gene of such an antibody.

In one embodiment of the present invention, a PC antibody binds to an epitope that overlaps with (or is identical to) the monoclonal antibody 11B7. In the present invention, such an antibody is referred to as an “antibody that binds to a substantially identical site”. For example, an antibody which binds to a site substantially identical to a site in PC to which a monoclonal antibody described in the Examples binds, can be obtained by analyzing epitopes of the above-described monoclonal antibody using a known method of epitope mapping using partial PC carbohydrate or the like, and then preparing antibodies that bind to a carbohydrate comprising the identified epitope, which is used as an antigen. Such an antibody is expected to comprise a suppressing activity similar to that of the PC antibodies. Competitive assays, for example, can be used to determine whether or not two antibodies bind to a substantially identical site on an antigen. Specifically, when the binding of the first anti-PC antibody with PC is competitively inhibited by the second anti-PC antibody, the first antibody and the second antibody can be judged to bind to a substantially identical site in the antigen. Thus, the present invention includes antibodies which bind to a site substantially identical to a site in PC to which an antibody isolated in the Examples binds.

The antibodies of the present invention also include antibodies which comprise the complementarity-determining region (CDRs) of the monoclonal antibody 11B7, or complementarity-determining regions functionally equivalent thereto. The term “functionally equivalent” refers to comprising amino acid sequences similar to the amino acid sequences of CDRs of any of the monoclonal antibodies isolated in the Examples. The term “CDR” refers to a region in an antibody variable region (also called “V region”), and determines the specificity of antigen binding. The H chain and L chain each have three CDRs, designated from the N terminus as CDR1, CDR2, and CDR3. There are four regions flanking these CDRs: these regions are referred to as “framework,” and their amino acid sequences are highly conserved. The CDRs can be transplanted into other antibodies, and thus a recombinant antibody can be prepared by combining CDRs with the framework of a desired antibody. One or more amino acids of a CDR can be modified without losing the ability to bind to its antigen. For example, one or more amino acids in a CDR can be substituted, deleted, and/or added.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and preferably within 35%, and still more preferably within 30% (e.g., within 25%). The identity of amino acid sequences can be determined as described herein.

In the present invention, recombinant antibodies artificially modified to reduce heterologous antigenicity against humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, is obtained by substituting an H or L chain complementarity determining region (CDR) of an antibody of a nonhuman mammal such as a mouse, with the CDR of a human antibody. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

Methods for obtaining human antibodies are also known. For example, desired human antibodies with antigen-binding activity can be obtained by (1) sensitizing human lymphocytes with antigens of interest or cells expressing antigens of interest in vitro; and (2) fusing the sensitized lymphocytes with human myeloma cells such as U266 (see Examined Published Japanese Patent Application No. (JP-B) Hei 1-59878). Alternatively, the desired human antibody can also be obtained by using an antigen to immunize a transgenic (Tg) animal that comprises a partial or entire repertoire of human antibody genes (see Nature Genetics 7:13-21 (1994); Nature Genetics 15:146-156 (1997); Nature 368:856-859 (1994); International Patent Application WO 93/12227, WO 92/03918, WO 94/02602, WO 94/25585, WO 96/34096, and WO 96/33735). Specifically, such Tg animals are created as follows: a nonhuman mammal in which the loci of heavy and light chains of an endogenous immunoglobulin have been disrupted, and instead, the loci of heavy and light chains of a human immunoglobulin have been introduced via Yeast artificial chromosome (YAC) vectors and the like, is obtained by creating knockout animals or Tg animals, or mating such animals. The immunoglobulin heavy chain loci can be functionally inactivated, for example, by introducing a defect at a certain site in a J region or C region (e.g., Cμ region). The immunoglobulin light chains (e.g., κ chain) can be functionally inactivated, for example, by introducing a defect at a certain site in a J region or C region, or a region comprising the J and C regions.

Such a humanized antibody can also be obtained from culture supernatant, by using genetic engineering technology to transform eukaryotic cells with cDNAs that encode each of the heavy and light chains of the antibody, or preferably vectors comprising these cDNAs, and then culturing the transformed cells that produce the recombinant human monoclonal antibody. The hosts are, for example, desired eukaryotic cells, preferably mammalian cells, such as CHO cells, lymphocytes, and myelomas.

Furthermore, techniques to obtain human antibodies by panning with a human antibody library are known. For example, the variable region of a human antibody is expressed as a single chain antibody (scFv) on the surface of a phage, using phage display method, and phages that bind to the antigen can be selected. By analyzing the genes of selected phages, the DNA sequences encoding the variable regions of human antibodies that bind to the antigen can be determined. If the DNA sequences of scFvs that bind to the antigen are identified, appropriate expression vectors comprising these sequences can be constructed, and then introduced into appropriate hosts and expressed to obtain human antibodies. Such methods are already well known (see WO 92/01047, WO 92/20791, WO 93/06213, WO 93/11236, WO 93/19172, WO 95/01438, and WO 95/15388).

When the antibody genes have been isolated and introduced into an appropriate host, hosts and expression vectors can be used in appropriate combination to produce the antibodies. As eukaryotic host cells, animal cells, plant cells, and fungal cells may be used. The animal cells include: (1) mammalian cells such as CHO, COS, myeloma, baby hamster kidney (BHK), HeLa, and Vero cells; (2) amphibian cells such as Xenopus oocytes; or (3) insect cells such as sf9, sf21, and Tn5, or silkworms. Known plant cells include cells derived from the Nicotiana genus such as Nicotiana tabacum, which can be callus cultured. Known fungal cells include yeasts such as the Saccharomyces genus, for example Saccharomyces cerevisiae, and filamentous fungi such as the Aspergillus genus, for example Aspergillus niger. Prokaryotic cells can also be used in production systems that utilize bacterial cells. Known bacterial cells include E. coli and Bacillus subtilis. The antibodies can be obtained by transferring the antibody genes of interest into these cells using transformation, and then culturing the transformed cells in vitro.

The isotypes of the antibodies of the present invention are not limited. The isotypes include, for example, IgG (IgG1, IgG2, IgG3, and IgG4), IgM, IgA (IgA1 and IgA2), IgD, and IgE. The antibodies of the present invention may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment, and contains a complete antigen recognition site and a binding site. This region is a dimer (V_{H}-V_{L} dimer) wherein the variable regions of each of the heavy chain and light chain are strongly connected by a noncovalent bond. The three CDRs of each of the variable regions interact with each other to form an antigen-binding site on the surface of the V_{H}-V_{L} dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, a preferred antibody fragment of the present invention is an Fv fragment, but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′)₂. Other chemically crosslinked antibody fragments are also known to those skilled in the art. Pepsin digestion of an antibody yields two fragments; one is a F(ab′)₂ fragment which comprises two antigen-binding domains and can cross-react with antigens, and the other is the remaining fragment (referred to as pFc′). Herein, an antibody fragment equivalent to that obtained by pepsin digestion is referred to as a “F(ab)-2-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′)₂-SH fragments can be recovered directly from hosts, such as E. coli, and then allowed to form F(ab′)₂ fragments by chemical crosslinking (Carter et al., Bio/Technology 10:163-167 (1992)). In an alternative method, F(ab′)₂ fragments can be isolated directly from a culture of recombinant hosts.

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (V_{L}) and a heavy chain variable region (V_{H}) in an identical chain are connected via a short linker, for example, a linker of about five residues, so that they cannot bind together. Because the linker between the two is too short, the V_{L} and V_{H} in the same polypeptide chain cannot form a single chain V region fragment, but instead form a dimer. Thus, a diabody has two antigen-binding domains. When the V_{L} and V_{H} regions against the two types of antigens (a and b) are combined to form V_{La}-V_{Hb} and V_{Lb}-V_{Ha} via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, preferably, a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

The antibodies of the present invention described above can be used in a passive immunity treatment of an individual that has been, or is suspected of being, infected with F. tularensis.

### V. Nucleic Acids Encoding Antibodies

The present invention further provides nucleic acid sequences that encode the antibodies described above.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

The terms “protein,” “peptide” and “polypeptide” are used interchangeably herein.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

The term “chimeric” refers to any gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, N.Y. (1993). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

### VI. Formulations and Methods of Administration

The PC vaccines and PC passive immunity compositions (e.g., anti-PC antibodies) of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following example is intended to illustrate but not limit the invention.

### Example 1

### Identification, Characterization and Immunogenicity of an O-Antigen of the Capsular Polysaccharide of Francisella tularensis

There has been heightened interest in understanding the basic biology and pathogenesis of Francisella tularensis because of the potential to use this organism as a bioterroism weapon. The availability of a safe and effective subunit vaccine for prevention of infections caused by F. tularensis would be important in protecting human populations against this risk. Investigators have searched for a capsular structure from F. tularensis for over forty years. This Example describes the isolation and characterization of a conserved O-antigen capsular polysaccharide of F. tularensis. This capsular polysaccharide is a high molecular weight structure composed of repeating polymers of the organism's O-antigen subunit. Vaccination of mice with this capsular polysaccharide can protect against at least 100 times the lethal dose of this organism.

Francisella tularensis is a gram-negative, aerobic, facultative intracellular bacterium and is the etiological agent of tularemia. The organism was first described by McCoy and Corbin in 1911 in Tulare County California (McCoy G, CW C (1912), Further observations on a plague-like diseases of rodents with preliminary note on the causative agent. J Infect Dis 10: 61-72). F. tularensis is found throughout the Northern hemisphere. Infection with F. tularensis can occur by inhalation, insect bite, subcutaneous inoculation through a break in the skin, ingestion of contaminated meat or water, or by animal bite (Cross A S, Calia F M, Edelman R (2007) From rabbits to humans: the contributions of Dr. Theodore E. Woodward to tularemia research. Clin Infect Dis 45 Suppl 1: S61-67). F. tularensis is one of the most infectious bacterial organisms known and as few as 10 organisms can cause disease in humans by inoculation or inhalation (Saslaw S, Eigelsbach H T, Prior J A, Wilson H E, Carhart S (1961) Tularemia vaccine study. Respiratory challenge. Arch Intern Med 107: 702-714; Saslaw S, Eigelsbach H T, Wilson H E, Prior J A, Carhart S (1961) Tularemia vaccine study. Intracutaneous challenge. Arch Intern Med 107: 689-701). In the United States, the majority of endemic disease occurs in hunters, laboratory personnel and children in rural areas. The highest incidence of disease has occurred in the USA over the past decade in Missouri and Arkansas (Anonymous (2002) Tularemia—United States, 1990-2000. MMWR 51: 181-184). Children from age three to ten and adults over 50 have the greatest incidences of disease (Anonymous (2002) Tularemia—United States, 1990-2000. MMWR 51: 181-184). Several forms of the disease can occur that depend on the route of infection, dose of bacteria and virulence of the infecting organism, including: ulceroglandular, glandular, oculoglandular, oropharyngeal and pneumonic. Infection with F. tularensis is marked with abrupt onset of symptoms, including fever, headache and body aches (Cross A S, Calia F M, Edelman R (2007), From rabbits to humans: the contributions of Dr. Theodore E. Woodward to tularemia research. Clin Infect Dis 45 Suppl 1: S61-67). Left untreated, infection is associated with high morbidity and mortality.

F. tularensis is classified as a Category A biological agent by the Strategic Planning Work Group of the Centers for Disease Control and Prevention as most likely to pose a potential national security risk (Anonymous (2002) Tularemia—United States, 1990-2000. MMWR 51: 181-184; Dennis D T, Inglesby T V, Henderson D A, Bartlett J G, Ascher M S, et al. (2001) Tularemia as a biological weapon: medical and public health management. Jama 285: 2763-2773). F. tularensis has the potential to be an efficient agent of biological warfare because it is highly infectious (Dennis D T, Inglesby T V, Henderson D A, Bartlett J G, Ascher M S, et al. (2001) Tularemia as a biological weapon: medical and public health management. Jama 285: 2763-2773). In addition, the bacterium is stable over a variety of environmental conditions, is easily dispersed as an aerosol, large quantities of the bacterium can be easily manufactured, the general population is susceptible to infection and infections lead to high morbidity and mortality (Kaufmann A F, Meltzer M I, Schmid G P (1997) The economic impact of a bioterrorist attack: are prevention and postattack intervention programs justifiable? Emerg Infect Dis 3: 83-84). It is estimated that intentional airborne release of F. tularensis into a metropolitan area of a major city would result in major morbidity, mortality and financial loss (Kaufmann A F, Meltzer M I, Schmid G P (1997). The economic impact of a bioterrorist attack: are prevention and postattack intervention programs justifiable? Emerg Infect Dis 3: 83-84).

Previously, a live attenuated F. tularensis vaccine was available to at-risk personnel; however, it did not provide complete protection against all forms of the disease. Vaccinated human volunteers were protected during aerosol infection from the most dangerous typhoidal form of infection, but the incidence of the ulceroglandular form of the disease was not affected; instead, vaccination lessened the severity of the infection (Tarnvik A, Henning C, Falsen E, Sandstrom G (1989) Isolation of Francisella tularensis biovar palaearctica from human blood. Eur J Clin Microbiol Infect Dis 8: 146-150). Development of a new vaccine is necessary because of limitations with the current vaccine. These include difficulty in standardizing the vaccine because it is administered via scarification and phenotypic instability of the vaccine strain. Little is known about the necessary protective antigens or what arm of the immune response should be targeted with the vaccine. Recent reports indicate that the vaccine strain may be able to reacquire some virulence characteristics on passage (Cherwonogrodzky J W, Knodel M H, Spence M R (1994) Increased encapsulation and virulence of Francisella tularensis live vaccine strain (LVS) by subculturing on synthetic medium. Vaccine 12: 773-775). Therefore, an intensified search is underway to develop a defined subunit vaccine comprised of Francisella cell surface components such as protein antigens, lipopolysaccharide (LPS) (Nierengarten M B, Lutwick L I (2002), Developing New Tularemia Vaccines. Medscape Infectious Diseases 4) and/or capsule, or a live vaccine with specific genetic modifications which preclude reversion to virulence.

Capsular antigens have been proven to be the basis for effective vaccines for Gram-negative and Gram-positive human pathogens (Artenstein M S, Gold R, Zimmerly J, Wyle F A, Schneider H, et al. (1976) Prevention of meningococcal disease by group C polysaccharide vaccine. NEJM 282: 417-420; Adamkiewicz T V, Silk B J, Howgate J, Baughman W, Strayhom G, et al. (2008) Effectiveness of the 7-valent pneumococcal conjugate vaccine in children with sickle cell disease in the first decade of life. Pediatrics 121: 562-569). F. tularensis has been considered to be encapsulated for over 40 years based on smooth to rough colony phenotype transition and evidence of the presence of conserved polysaccharide structures in the organism (Carlisle H N, Hinchcliffe V, Saslaw S (1962) Immunodiffusion studies with Pasturella tularensis antigen-rabbit antibody systems. J Immunol 89: 638-644). Currently, little is known about the chemical structure or antigenic nature of the capsular antigen of F. tularensis. A purified capsular preparation has never been described and most published reports dealing with capsule depended on identifying “rough” colonies which were considered unencapsulated (Sorokin V M, Pavlovich N V, Prozorova L A (1996) Francisella tularensis resistance to bactericidal action of normal human serum. FEMS Immunol Med Microbiol 13: 249-252; Sandstrom G, Lofgren S, Tarnvik A (1988) A capsule-deficient mutant of Francisella tularensis LVS exhibits enhanced sensitivity to killing by serum but diminished sensitivity to killing by polymorphonuclear leukocytes. Infect Immun 56: 1194-1202).

Using a crude “capsular” extract from F. tularensis subsp. tularensis SCHU S4 as prepared by the method of Hood as an immunogen (Hood A M (1977) Virulence factors of Francisella tularensis. J Hyg (Lond) 79: 47-60), we developed a bank of monoclonal antibodies and evaluated each for binding to antigens with the characteristics of a typical polysaccharide capsule that help distinguish it from the LPS. The characteristics include being protease and phenol resistant, ethanol precipitable and having a high molecular weight (>100 kDa). Two of these monoclonal antibodies bound a high mass capsule-like structure on Western blot that matched these characteristics. Using one of these antibodies as a probe, we were able to purify this capsular material, free of other bacterial components including LPS. Physiochemical analysis of the antigen indicated that it contains a tetrasaccharide repeat, 2-acetamido-2,6-dideoxy-O-D-glucose (O-QuiNAc); 4,6-dideoxy-4-formamido-D-glucose (O-Qui4NFm), and 2-acetamido-2-deoxy-O-D-galacturonamide (O-GalNAcAN) of similar, if not identical, structure and composition to the LPS O-antigen. Compositional analysis and mass spectrometry studies did not identify Kdo or lipid A as a component of the capsular antigen. Passive immunization with this monoclonal antibody and active immunization of BALB/c mice with the capsule was protective against a challenge of 150 fold the LD₅₀ of F. tularensis LVS. Immunization of these mice with the capsule resulted in generation of antibodies to the capsule preparation and not the O-antigen subunits of the LPS demonstrating a unique epitopic structure to the capsule. These results indicate that the F. tularensis capsule produces an O-antigen capsule which may have the potential to be a protective immunogen against F. tularensis infection.

Results

Development and Characterization of the F. tularensis Anti-Capsular Monoclonal Antibody

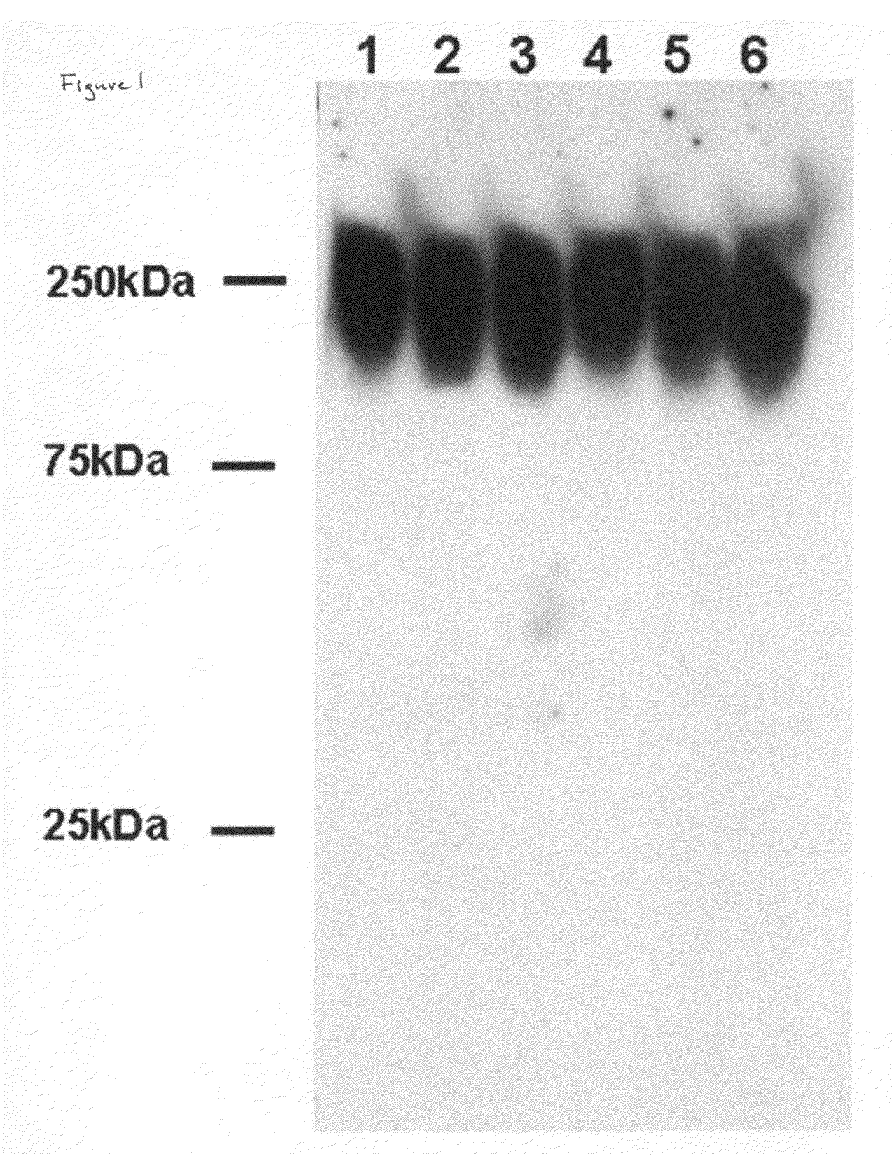

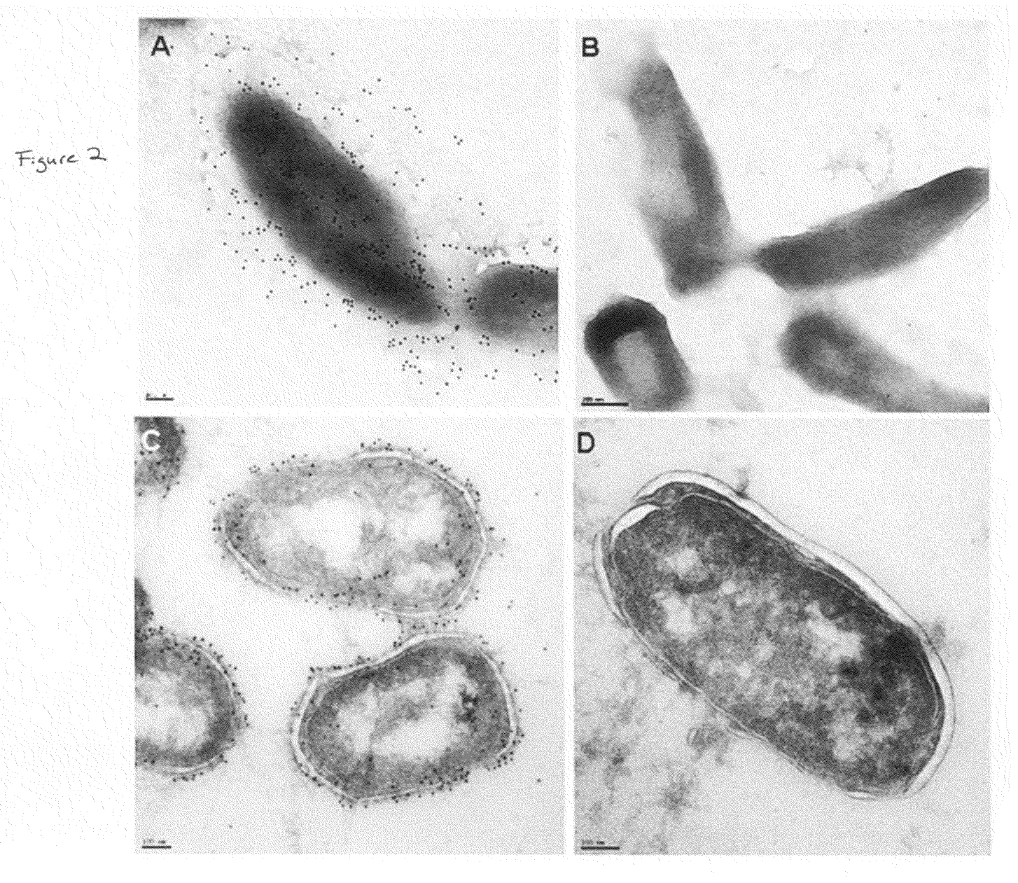

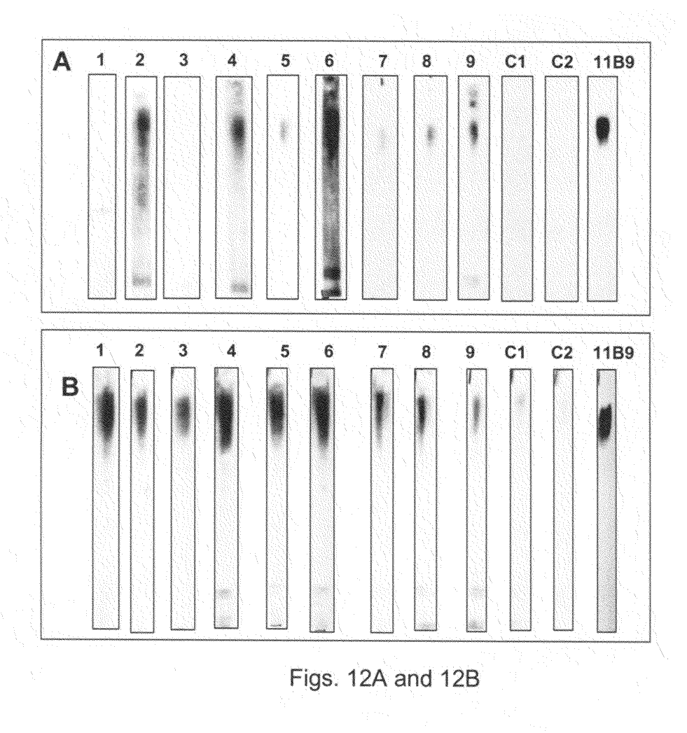

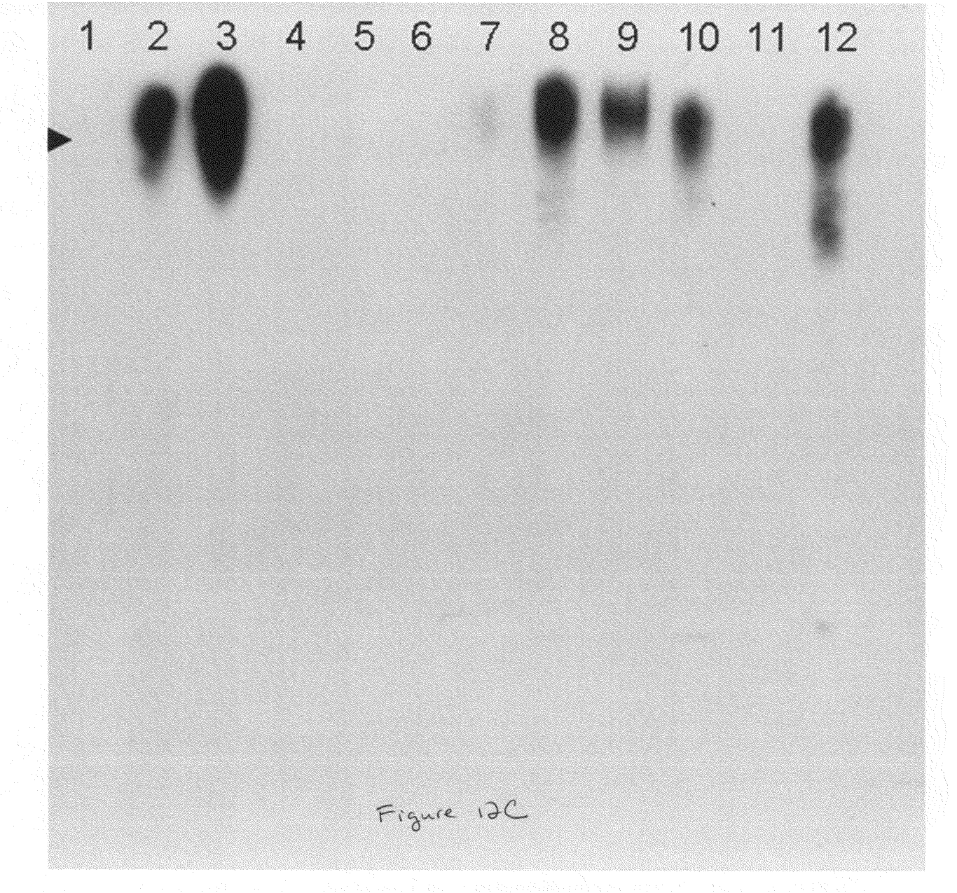

In order to determine if F. tularensis SCHU S4 produced a capsular antigen and to develop a probe for it, we elected to make monoclonal antibodies (MAb) to a high salt extract of F. tularensis subsp. tularensis SCHU S4 prepared according to the method of Hood (Hood A M (1977) Virulence factors of Francisella tularensis. J Hyg (Lond) 79: 47-60). This resulted in the identification of antibody 11B7 which recognized a structure with the characteristics of a carbohydrate capsular antigen, i.e., high mass, diffusely migrating, and resistant to proteolytic enzymes and phenol. As can be seen in FIG. 1, Western blot analysis indicated that MAb 11B7 bound to a structure between 100 kDa and 250 kDa. Subsequent studies demonstrated that this structure had the characteristics of a polysaccharide capsule. FIG. 1 also demonstrates the conserved nature of the putative capsule as five distinct strains of F. tularensis obtained as glutaraldehyde-fixed organisms and F. tularensis subsp. holarctica 1547 reacted to this antibody. Our studies demonstrated that the capsular material was firmly associated with the intact bacteria as only minimal amounts of capsule could be detected in broth culture supernates cleared of organisms at 4, 8 and 24 hours (FIG. 12C). Subsequent studies indicated that a similar structure could be identified in 15 F. tularensis type A and type B strains from a wide geographic distribution in North America (Table 1). We performed immunoelectron microscopy using “whole mount” samples and cryo-immunoelectron microscopy on strains F. tularensis SCHU S4, 1547 and LVS using MAb 11B7. A representative stained images of each is shown in FIGS. 2A and 2C. This demonstrates the presence of circumferential labeling of the organism by this antibody confirming the surface location of the antigen. The secondary antibody controls for both studies show no labeling (FIGS. 2B and 2D).

TABLE 1
Reactivity of F. tularensis strains and
mutants with 11B7 and anti-LPS MAb FB11

| | Capsule¹ | LPS² |
|---|---|---|
| Strain | positive | O-antigen |
| F. tularensis SHU S4 | Yes | Yes |
| F. tularensis LVS-FDA | Yes | Yes |
| F. tularensis LVS-VT | Yes | Yes |
| F. tularensis 1547 | Yes | Yes |
| F. tularensis 1547msbB | Yes | No |
| F. tularensis l547msbB [pmsbB] | Yes | Yes |
| F. tularensis 0673-0674 | Yes | No |
| F. tularensis 1623 | Yes | Yes |
| F. tularensis T1 0902 | Yes | Yes |
| F. tularensis WY96-3418 | Yes | Yes |
| F. tularensis MA00-2987 | Yes | Yes |
| F. tularensis NR-50(NIH B-38) | Yes | Yes |
| F. tularensis OSPHL 2001-1011 | Yes | Yes |
| F. tularensis OSPHL 2001-1-0513 | Yes | Yes |
| F. tularensis OSPHL 2002-1-0990 | Yes | Yes |
| F. tularensis OSPHL 2001-1-0074 | Yes | Yes |
| F. tularensis OSPHL 2001-1-0143 | Yes | Yes |
| F. tularensis LVScapB | Yes | Yes |
| F. tularensis LVScapC | Yes | Yes |
| F. tularensis LVSwbtC | No | No |
| F. tularensis LVSwbtK | Yes | Modified* |
| F. tularensis LVS0708 | No | Yes |
| F. tularensis LVSwbtM | No | No |
| F. tularensis LVSwbtK | No | No |
| F. tularensis LVSwbtA1 | No | No |
| F. tularensis LVSwbtA2 | No | No |
| F. tularensis LVSwbtI | No | No |
| F. tularensis LVS FTT0706 | Yes | No |
| F. tularensis SCHU S4 0673-0674 | Yes | No |
| F. novicida U112 | No | No |

*high mass O-antigens absent compared to parent strain.
¹Reacted with MAb 11B7 to high mass structure on Western blot
²Reacted with MAb FB11 demonstrating O-antigen subunits

Characterization of the Capsule from Francisella tularensis

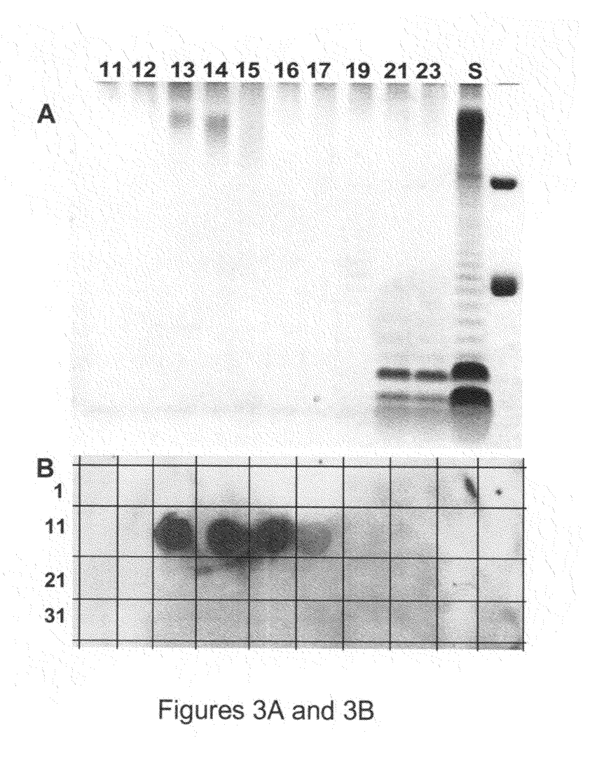

Using the NaCl extraction method described by Hood (Hood A M (1977) Virulence factors of Francisella tularensis. J Hyg (Lond) 79: 47-60), followed by proteinase digestion, phenol extraction, Triton X-114 treatment and Sephacryl S500 chromatography, we have been able to isolate the capsule away from contaminating LPS. FIG. 3A shows a ProQ Emerald glycostain of fractions from a Sephacryl S-500HR chromatography of a F. tularensis Schu S4 crude capsule prep before treatment with Triton X114. The buffer used contained 100 mM NaCl, 10 mM EDTA, 10 mM Tris, 2% SDS pH 7.4 and the chromatography was performed at 37° C. The column void volume was 11 ml (tube 11) and the bed volume was 30 ml. The capsule eluted between 12 and 16 ml and the LPS eluted at 21 ml. FIG. 3B shows an immunodot assay of each fraction using MAb 11B7. The MAb 11B7 reactivity to capsule occurs in tubes 12 through 15. Using MAb 11B7 to track the capsule, we have been able to isolate highly purified capsule in milligram quantities from F. tularensis Schu S4, F. tularensis LVS and F. tularensis subsp. holarctica strain 1547.

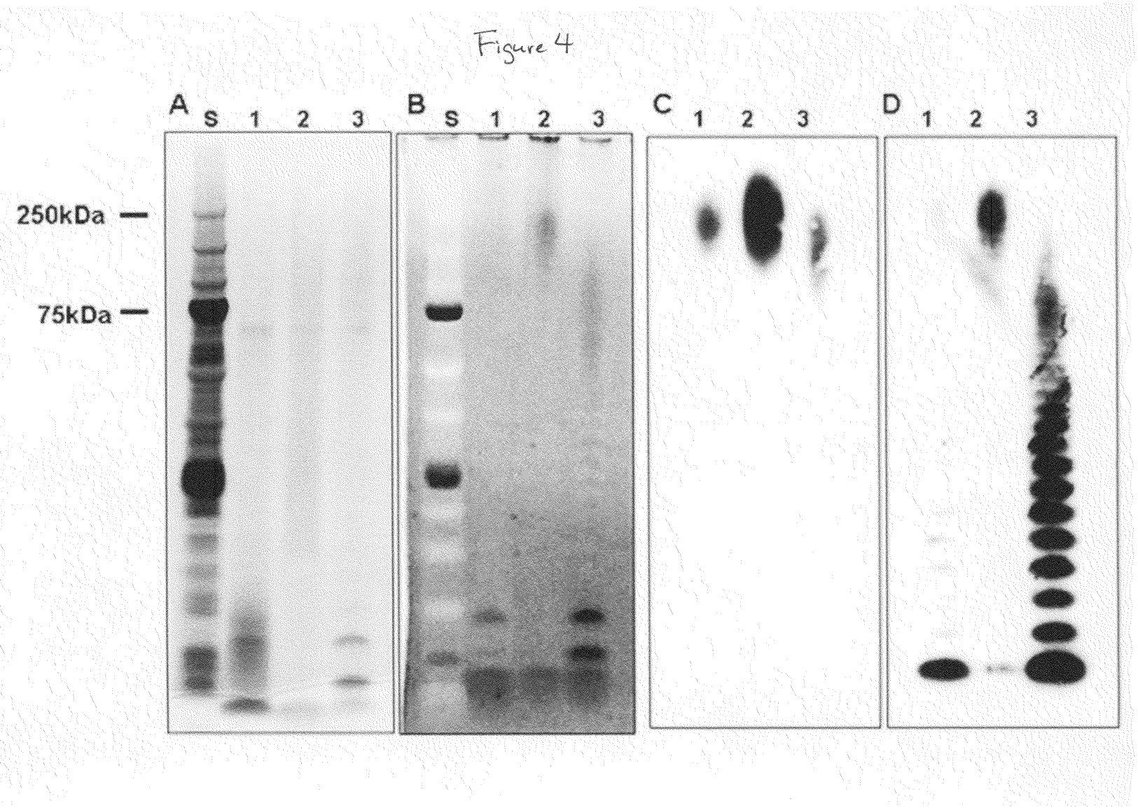

FIG. 4 shows comparative staining of the capsule and LPS with silver stain, ProQ Emerald glycostain, MAb 11B7 and the anti-LPS MAb FB11. As can be seen, the silver stain does not readily show the capsule which is visible in the ProQ Emerald stain. FIG. 4 Panel C shows that MAb 11B7 reacts most intensely with lane 2, indicating that this is where the majority of the capsule is found. Small amounts of low mass residual capsule are detected in the LPS preparation taken from the Triton X114 fraction (FIG. 4, Panel C, lane 3). The anti-LPS MAb FB11 reacts with both the high molecular weight capsule, and also the O-antigen of the LPS (FIG. 4, Panel D, lanes 2 and 3). The absence of a high molecular weight band reacting with this MAb in the LPS lane indicates that there is very little capsule material present in the LPS sample. The absence of the bands corresponding to the O-antigen repeating unit of the LPS in the capsule sample indicates that LPS is essentially absent from the final capsular preparation (FIG. 4, Panel D, lane 2).

Physicochemical Analysis of the F. tularensis Capsule

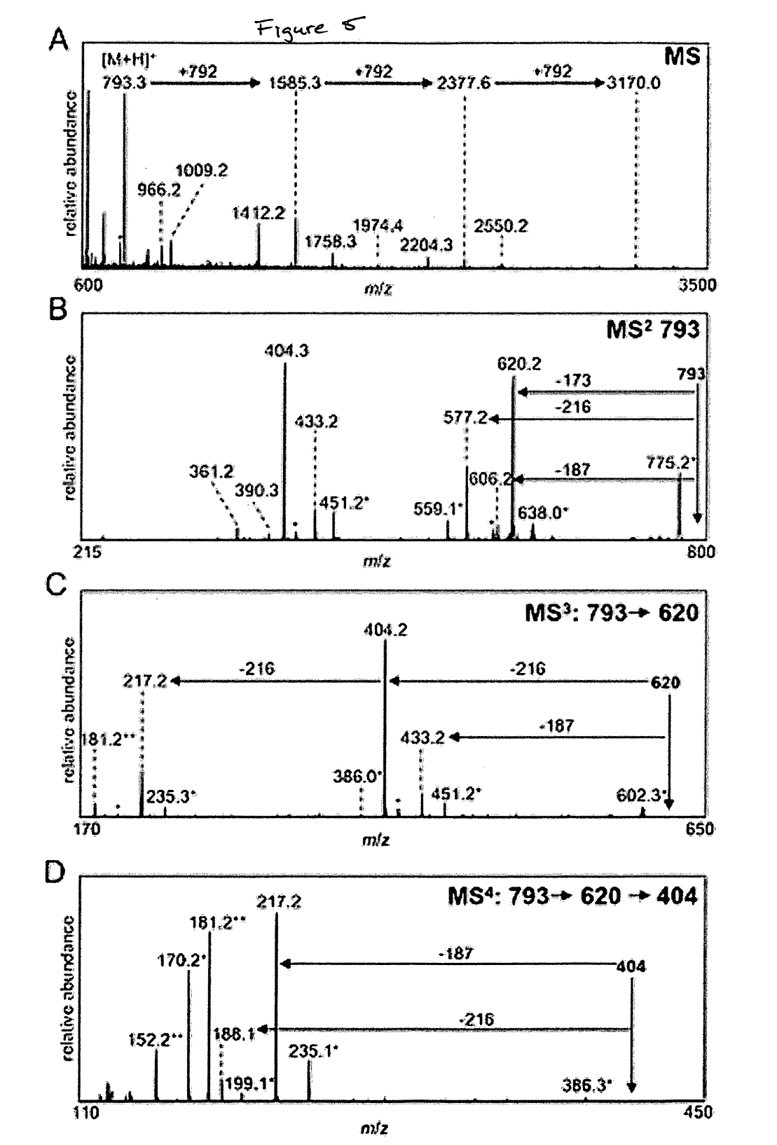

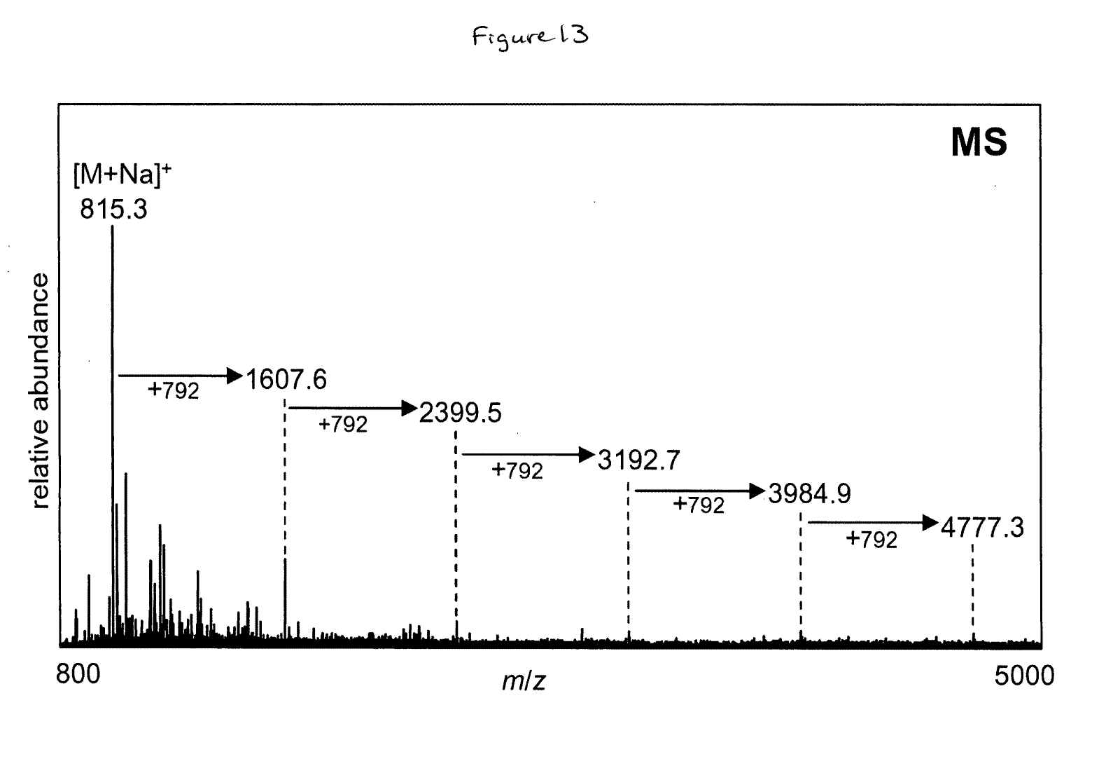

Compositional analyses of the capsule by MALDI-MS and GC-MS. The mass spectrometry (MS) approach we used to provide critical composition and sequence information on the capsule was based on methods we employed earlier for LPS and endotoxin (Phillips N J, Schilling B, McLendon M K, Apicella M A, Gibson B W (2004) Novel modification of lipid A of Francisella tularensis. Infect Immun 72: 5340-5348; Schilling B, McLendon M K, Phillips N J, Apicella M A, Gibson B W (2007) Characterization of lipid A acylation patterns in Francisella tularensis, Francisella novicida, and Francisella philomiragia using multiple-stage mass spectrometry and matrix-assisted laser desorption/ionization on an intermediate vacuum source linear ion trap. Anal Chem 79: 1034-1042). We examined the capsular material both directly and after limited HF treatment using matrix-assisted laser desorption ionization mass spectrometry (MALDI-MS) on an LTQ linear ion trap instrument coupled to an intermediate vacuum vMALDI ion source. In both cases, initial positive-ion MS data indicated that there was a 792 Dalton (Da) repeat unit. FIGS. 5A-5D show MALDI-MS analyses of unprocessed capsule in the positive ion mode. These data show that the predominant protonated species observed in the MS spectrum is one Da higher at m/z 793. In addition, monoisotopic masses observed at m/z 1585, 2377 and 3170, correspond to the addition of one, two or three of the 792 Da repeating units to the original structure, respectively. Data observed using MALDI-time of flight (MALDI-TOF) analyses, which allowed us to observe a larger mass range than the vMALDI, yielded similar results; a 792 Da repeating unit was observed, with data obtained for up to six repeating units (FIG. 13, Table 2).

TABLE 2
Proposed compositions of masses
observed by MALDI-TOF analyses

| [M + | [M + | |
|---|---|---|
| Na]⁺_{observed} | Na]⁺ | Proposed composition¹ |
| 815.32 | 815.31 | (GalNAcAN-GalNAcAN-QuiNAc-Qui4NFm)1 |
| 1607.56 | 1607.63 | (GalNAcAN-GalNAcAN-Qui NAc- Qui4N Fm)2 |
| 2399.47 | 2399.95 | (GalNAcAN-GalNAcAN-Qui NAc- Qui4N Fm)3 |
| 3192.66 | 3192.27 | (GalNAcAN-GalNAcAN-QuiNAc- Qui4NFm)4 |
| 3984.88 | 3984.59 | (GalNAcAN-GalNAcAN-Qui NAc- Qui4N Fm)5 |
| 4777.29 | 4776.91 | (GalNAcAN-GalNAcAN-Qui NAc- Qui4N Fm)6 |

¹GalNAcAN: 2-acetamido-2-deoxy-D-galacturonamide (M = 216.08 Da), QuiNAc: 2-acetamido-2,6-dideoxy-D-glucose (M = 187.09 Da), and Qui4N Fm: 4,6-dideoxy4-formamido-D-glucose (M = 173.07 Da). Masses listed are anhydro forms of each constituent.

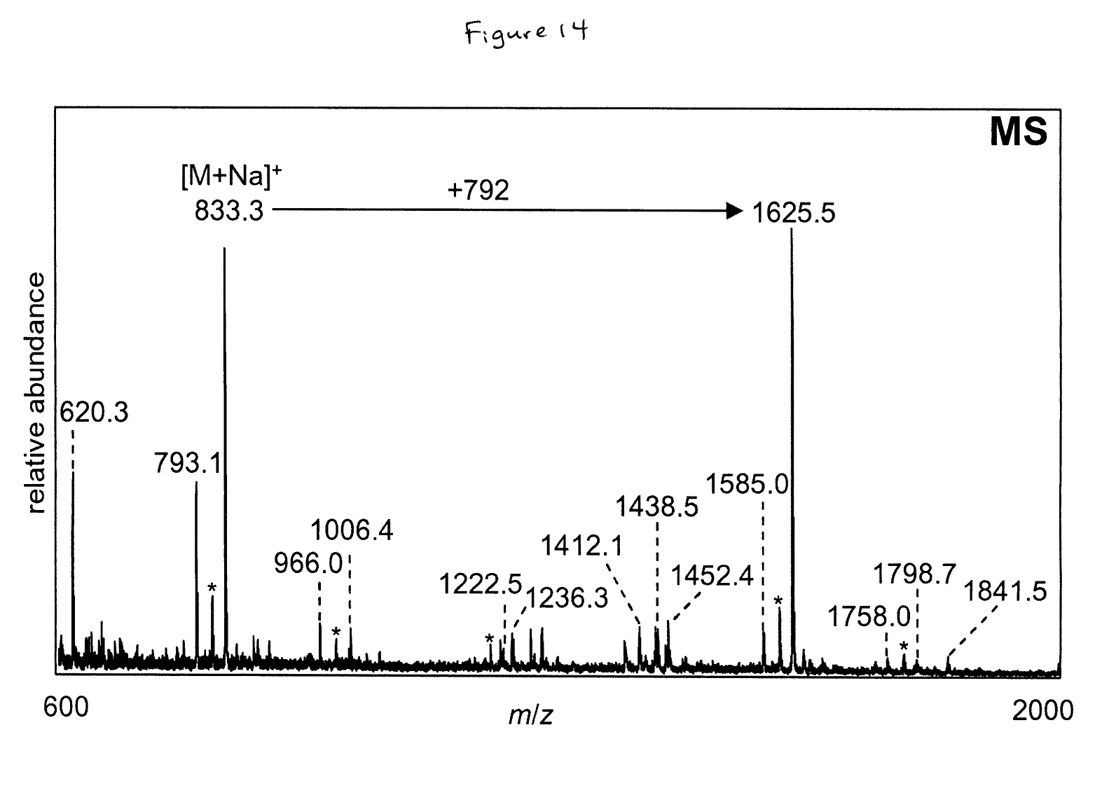

Similar to what was observed for the unprocessed capsule sample, MALDI-MS analyses of the limited HF-treated capsule also showed the presence of a 792 Da repeating unit (FIG. 14). In limited HF treatment the capsule underwent chemical hydrolysis to generate oligosaccharide fragments, so unlike the fragments generated in MALDI-MS in the unprocessed capsule which were derived from gas-phase fragmentation, the HF-treated capsule produced peaks shifted up in mass by the addition of water (M=792+18=810).

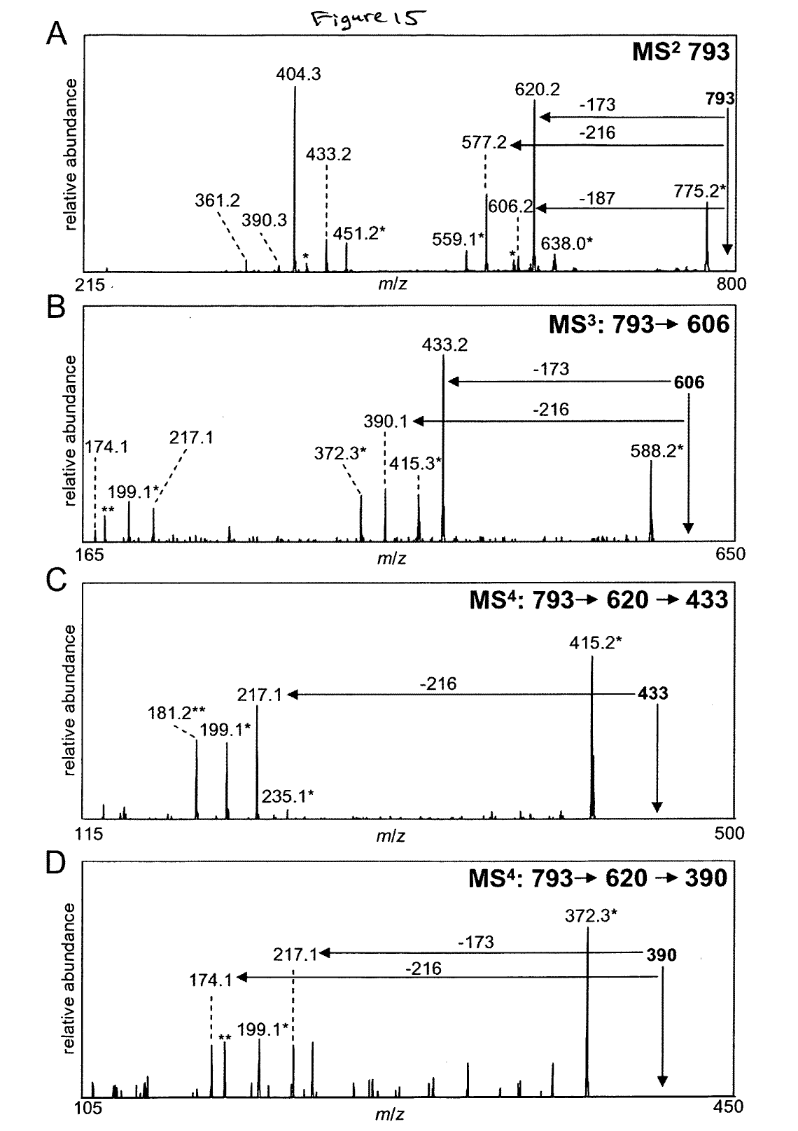

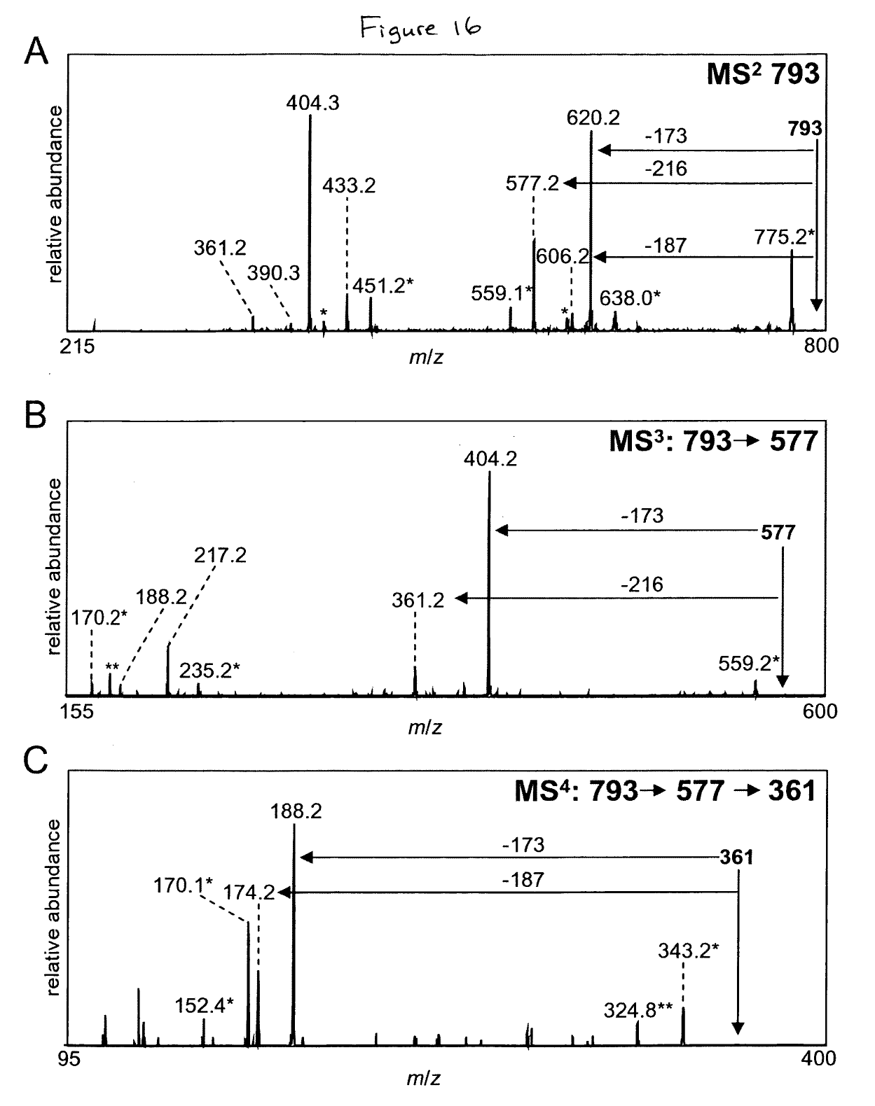

Multi-stage mass spectrometry (MSⁿ) of the unprocessed capsule was utilized to determine the individual components of the repeating unit (FIGS. 5A-5D). MS/MS analyses of the monoisotopic mass [M+H]⁺=793 generated two major fragments at m/z 620 and 404 and several minor fragments at m/z 606, 577, 433, 390, and 361. The mass at m/z 620 corresponds to the loss of 173 Da from the m/z 793 precursor ion. MS³ fragmentation of the m/z 620 peak resulted in one major fragment at m/z 404 and two minor fragments at m/z 433 and 217. The fragment at m/z 404 corresponds to the loss of 216 Da from the m/z 620 precursor ion. The peak observed at m/z 433 corresponds to the loss of 187 Da from the m/z 620 precursor ion. MS⁴ fragmentation of the precursor ion at m/z 404 showed fragments that corresponded to the loss of either 187 or 216 Da. These data demonstrated that the 792 Da unit is a tetrasaccharide containing two 216 Da components, one 187 Da component, and one 173 Da component. Further analyses using MSⁿ of the various observed fragment ions originating from the protonated tetrasaccharide, [M+H]⁺=793, gave several disaccharide fragments that defined various nearest neighbor combinations: 216-216 (m/z 433), 187-216 (m/z 404), 173-216 (m/z 390), and 173-187 (m/z 361) (FIGS. 5A-5D, FIGS. 15 and 16, Table 3). These data demonstrated that the sequence of these constituents was either 187-173-216-216 or 216-216-173-187. This composition and sequence is consistent with the previously published O-antigen structure for this strain of F. tularensis (Vinogradov E V, Shashkov A S, Knirel Y A, Kochetkov N K, Tochtamysheva N V, et al. (1991) Structure of the O-antigen of Francisella tularensis strain 15. Carbohydr Res 214: 289-297).

TABLE 3
Proposed compositions of masses
observed by MALDI-MSⁿ analyses

| [M + | [M + | | | |
|---|---|---|---|---|
| H]⁺_{observed} | H]⁺_{calculated} | GalNAcAN¹ | QuiNAc¹ | Qui4NFm¹ |
| 793.17 | 793.32 | 2 | 1 | Qui4NFm¹ |
| 620.17 | 620.17 | 2 | 1 | Qui4NFm¹ |
| 606.17 | 606.23 | 2 | 0 | Qui4NFm¹ |
| 577.17 | 577.24 | 1 | 1 | Qui4NFm¹ |
| 433.17 | 433.16 | 2 | 0 | Qui4NFm¹ |
| 404.25 | 404.17 | 1 | 1 | Qui4NFm¹ |
| 390.25 | 390.15 | 1 | 0 | Qui4NFm¹ |
| 361.17 | 361.16 | 0 | 1 | Qui4NFm¹ |

¹GalNAcAN: 2-acetamido-2-deoxy-D-galacturonamide (M = 216.08 Da), QuiNAc: 2-acetamido-2,6-dideoxy-D-glucose (M = 187.09 Da), and Qui4NFm: 4,6-dideoxy-4-formamido-D-glucose (M = 173.07 Da). Masses listed are dehydro forms of each constituent.

Compositional analyses of the capsule monosaccharides by high-pH anion exchange chromatography demonstrated that the capsule sugars were not common monosaccharides, as the major peaks seen in the capsule sample did not correlate with a standard mix of twelve standards (Tables 4 and 5).

TABLE 4
High pH-chromatography data from mixture of standards.

| | | | | Relative |
|---|---|---|---|---|
| | Ret. time | Height | Area | Area |
| Peak name | (min) | (nC) | (nC*min) | (%) |
| Fucose | 5.08 | 41.395 | 11.522 | 6.41 |
| Galactosamine | 10.17 | 74.981 | 36.301 | 20.19 |
| Glucosamine | 12.17 | 45.043 | 21.129 | 11.75 |
| Galactose | 13.42 | 25.916 | 11.888 | 6.61 |
| Glucose | 14.50 | 40.288 | 20.991 | 11.67 |
| Mannose | 16.08 | 13.781 | 9.973 | 5.55 |
| N-acetylneuraminic acid | 43.67 | 44.717 | 14.494 | 8.06 |
| ¹Kdo | 47.67 | 24.207 | 8.790 | 4.89 |
| Galacturonic acid | 52.50 | 5.459 | 1.847 | 1.03 |
| Glucuronic acid | 54.50 | 19.809 | 7.402 | 4.12 |
| N-glycolylneuraminic acid | 59.08 | 65.544 | 29.596 | 16.46 |
| ¹Iduronic acid | 61.17 | 11.753 | 5.893 | 3.28 |

Kdo: 3-deoxy-D-manno-octulosonic acid

TABLE 5
High pH-chromatography data from F. tularensis capsule.

| | | | | Relative |
|---|---|---|---|---|
| | Ret. time | Height | Area | Area |
| Peak name | (min) | (nC) | (nC*min) | (%) |
| Unknown | 4.17 | 120.134 | 108.367 | 18.43 |
| Unknown | 6.83 | 444.836 | 179.051 | 30.46 |
| Galactosamine | 10.17 | 22.212 | 8.765 | 1.49 |
| Glucosamine | 12.25 | 28.760 | 13.650 | 2.32 |
| Glucose | 14.50 | 15.189 | 7.612 | 1.29 |
| Unknown | 46.50 | 58.080 | 19.053 | 3.24 |
| Unknown | 50.00 | 534.195 | 219.448 | 37.33 |
| Unknown | 63.33 | 16.599 | 6.657 | 1.13 |
| Unknown | 69.58 | 51.572 | 25.270 | 4.30 |

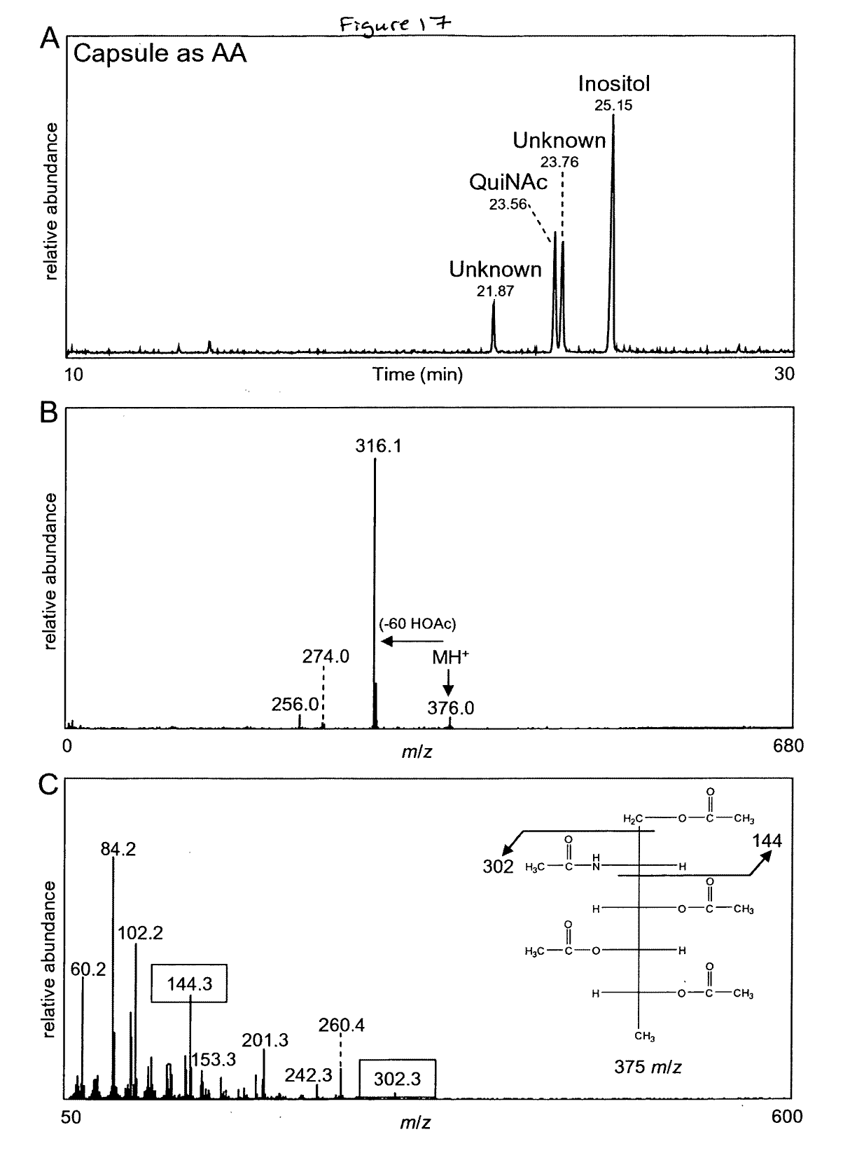

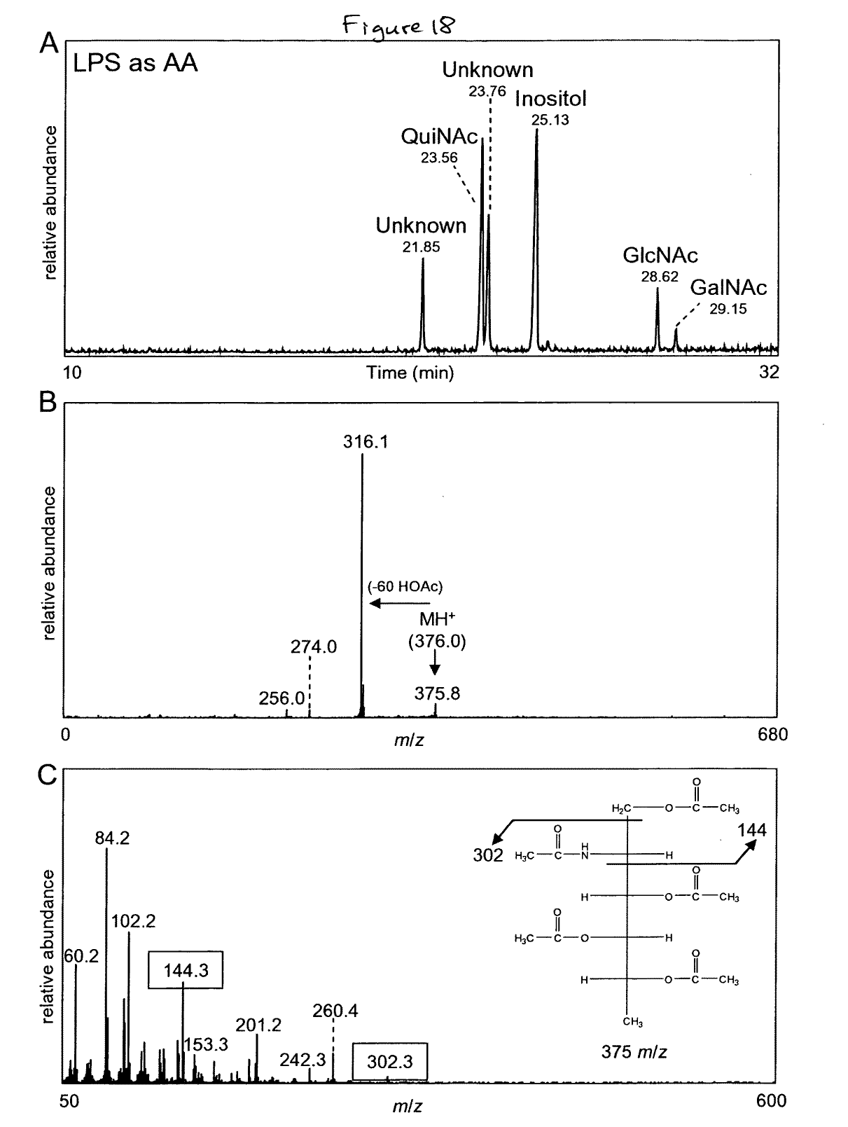

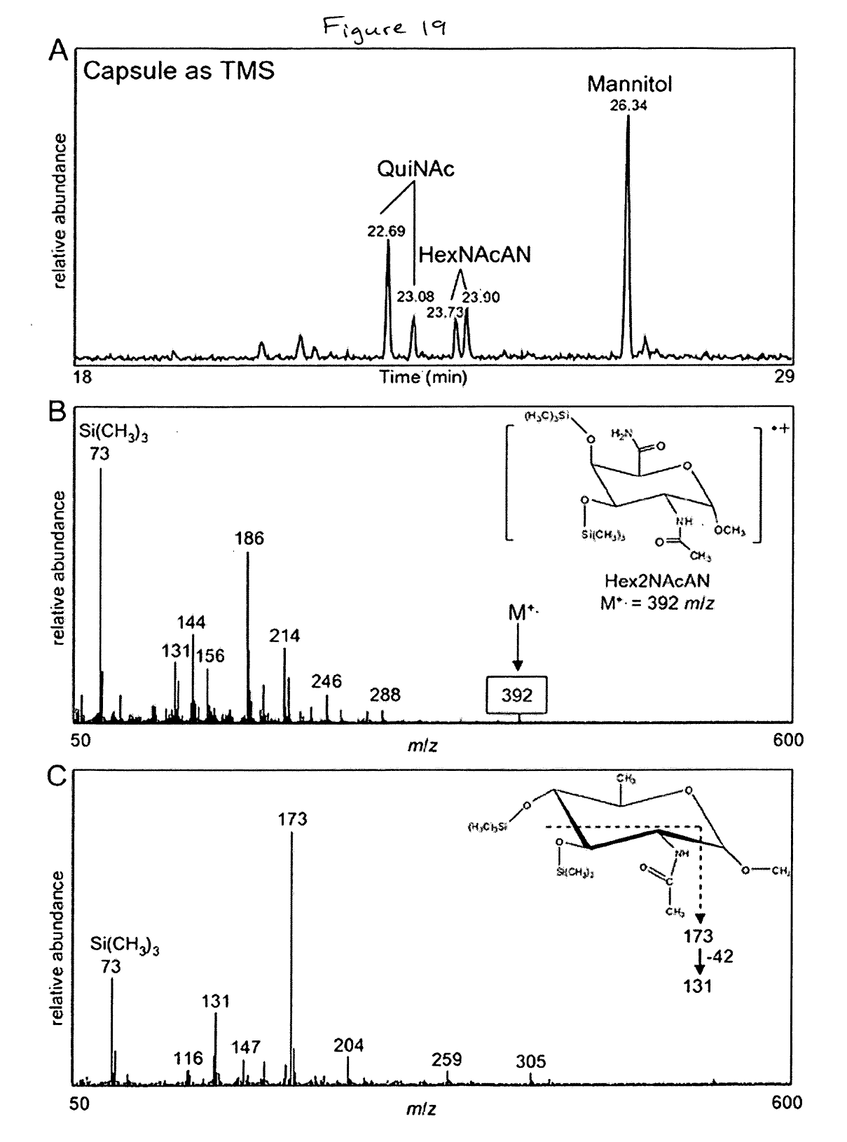

These data also showed that one of the LPS building blocks, Kdo, was not detected in the capsule sample. Further compositional analyses using GC-MS were also performed. GC-MS analyses of alditol acetate (AA) sugars from the capsule showed the presence of QuiNAc and two additional peaks which likely correspond to anhydro degradation products of Qui4NFm or QuiNAc (FIG. 17A). Assignments of the peaks were confirmed by both CI and EI analyses (FIGS. 17B-17C). Analyses of AA sugars of the LPS gave the same three peaks as the capsule sample, and additional LPS sugars, N-acetylglucosamine (GlcNAc) and N-acetylgalatosamine (GalNAc) (FIG. 18). GC-MS analyses of the capsule as a TMS derivative, confirmed the presence of QuiNAc and HexNAcAN in the sample (FIG. 19, Table 6). FIG. 19 shows the GC-MS analyses of TMS-derived LPS from the same strain of F. tularensis from which the capsule was extracted.

TABLE 6
Summary of GC-MS data for TMS derivatives
of F. tularensis capsule.

| | Ret. time | Relative Area |
|---|---|---|
| Peak name | (min) | (%) |
| ¹QuiNAc | 22.69 | 22.47 |
| ¹QuiNAc | 23.08 | 9.07 |
| ¹HexNAcAN | 23.73 | 8.08 |
| ¹HexNAcAN | 23.9 | 11.02 |
| ²Mannitol | 26.34 | 49.36 |

¹QuiNAc: 2-acetam ido-2,6-dideoxy-D-glucose, HexNAcAN: 2-acetam ido-2-deoxy-D-hexuronamide.
²Mannitol was included as an internal standard.

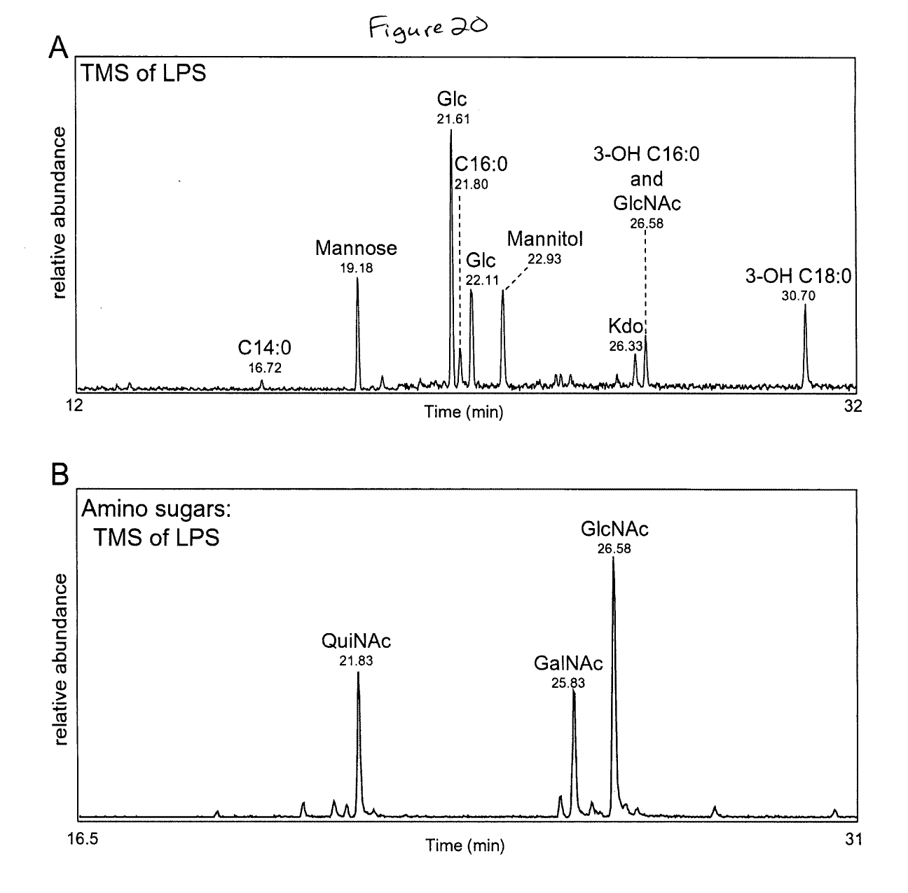

These data demonstrated that the LPS sample contained glucose, mannose, Kdo, GlcNAc, as well as components of the lipid A (C14:0, C16:0, 3-OH C16:0, and 3-OH C18:0) (FIG. 20, Table 7).

TABLE 7
Summary of GC-MS data for TMS derivatives
of F. tularensis LPS.

| | Ret. time | Relative Area |
|---|---|---|
| Peak name | (min) | (%) |
| Ribose | 13.32 | 1.04 |
| C14:0 | 16.72 | 1.32 |
| Mannose | 18.18 | 13.41 |
| Glucose | 21.61 | 29.81 |
| C16:0 | 21.8 | 5.53 |
| Glucose | 22.11 | 12 |
| ¹Mannitol | 22.93 | 13.06 |
| ²Kdo | 26.33 | 4.91 |
| ²GlcNAc, 3-OH C16:0 | 26.58 | 6.67 |
| 3-OH C18:0 | 30.7 | 12.26 |

¹Mannitol was included as an internal standard.
²Kdo: 3-deoxy-D-manno-octulosonic acid, GlcNAc: N-acetyl-glucosamine

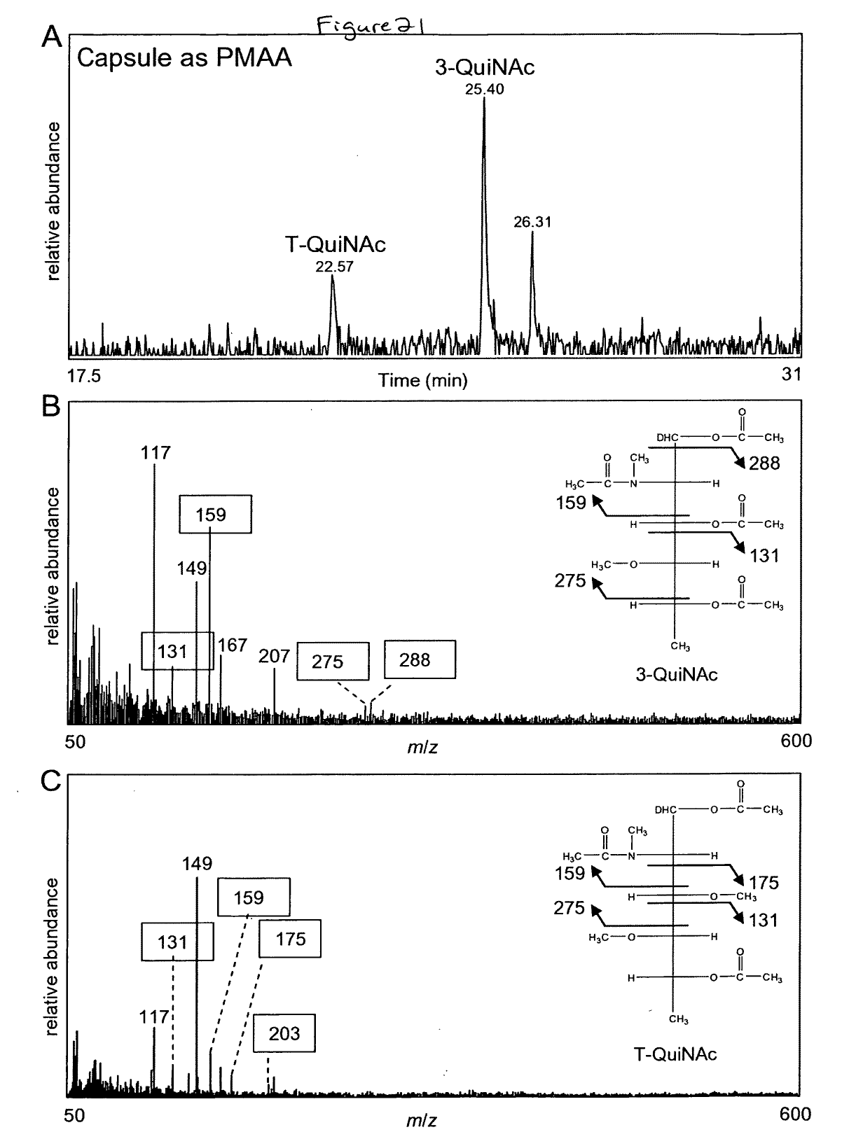

When these same samples were scanned for amino sugars, the presence of QuiNAc, GalNAc, and GlcNAc was also confirmed. Unlike the LPS sample, none of the LPS-specific components (Kdo or lipid A) were detected in the TMS-derived capsule sample. Partial linkage determination was performed by GC-MS analyses of partially methylated alditol acetates generated from capsule; these data demonstrated the presence of a terminal- and a 3-linked-QuiNAc in this structure (FIG. 21).

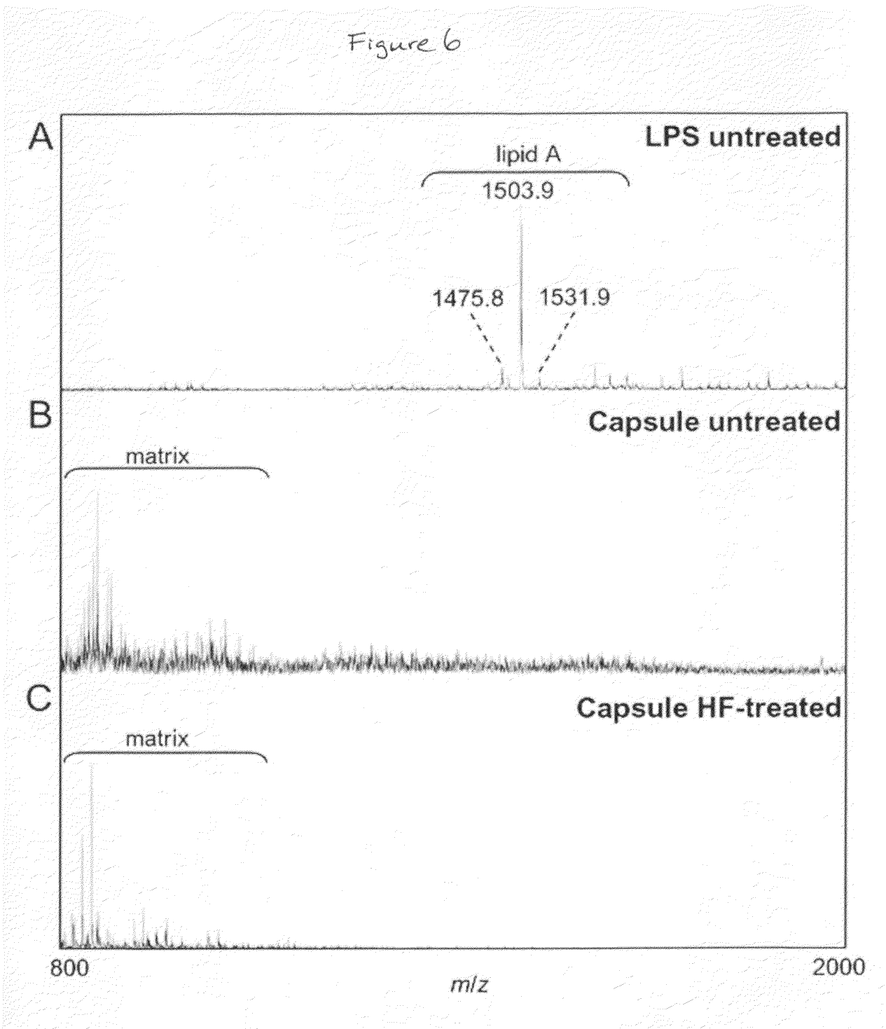

The present studies strongly suggest that the capsule contains the same carbohydrate repeating unit as the O-antigen but that it is a distinct structure from the LPS. Compositional analyses of the LPS and capsule showed that while both the Kdo and lipid A components were clearly present in the LPS sample, they were never detected in the capsule sample. In addition, negative-ion MALDI-MS data of unprocessed LPS and unprocessed and HF-treated capsule showed that the monoisotopic mass corresponding to the previously characterized F. tularensis tetraacyl lipid A, [M−H]⁻=1504 (Schilling B, McLendon M K, Phillips N J, Apicella M A, Gibson B W (2007) Characterization of lipid A acylation patterns in Francisella tularensis, Francisella novicida, and Francisella philomiragia using multiple-stage mass spectrometry and matrix-assisted laser desorption/ionization on an intermediate vacuum source linear ion trap. Anal Chem 79: 1034-1042), was readily detected in the LPS sample, whereas no masses corresponding to any species of F. tularensis lipid A were detected in any capsule samples (FIG. 6A-6C). MS′ analyses of the LPS sample confirmed that the peak at m/z 1504 was lipid A (data not shown). These data coupled with our genetic, chemical, and antibody specificity studies indicate that the capsule is distinct from the F. tularensis LPS even though both share the same O-antigen subunit structure.

NMR studies. NMR spectra were analyzed to determine if assignments of the capsular extract could be made that are consistent with the carbohydrate composition of the polysaccharide as determined by mass spectrometry. All data are related back to a ¹³C-HMQC experiment. Spin systems were built up starting from the anomeric position and extended sequentially using COSY and short (20 ms) mixing time TOCSY data. A longer (60 ms) mixing time TOCSY data set was used to both extend and further verify sequential connectivities. Connections between spin systems were made using a combination of NOESY data correlating spins near one another in space as well as HMBC to make through bond connections to spins across the glycosidic linkage.

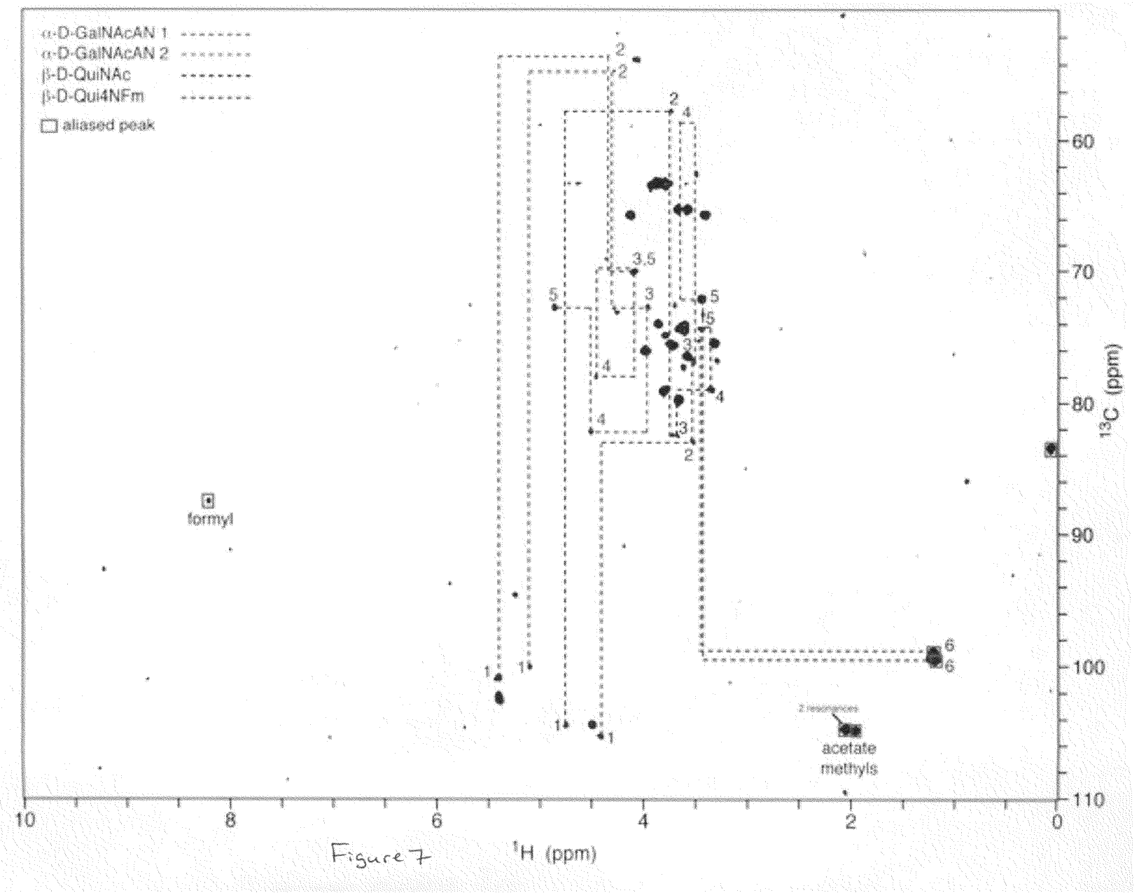

Analysis of mass spectrometric data indicated a 4 subunit repeat with masses arranged either 216-216-173-187 or 187-173-216-216, as the MS analysis data did not differentiate the reducing and non-reducing ends. There is little data consistent with the first model: no inter-ring HMBC crosspeaks, a single cross-glycosidic NOE of dubious merit, and only three other weak NOEs. However, there is a fair amount of NMR data consistent with the second model, which has a proposed composition 4)-α-D-GalNAcAN-(1->4)-α-D-GalNAcAN-(1->3)-β-D-QuiNAc-(1->2)-β-D-Qui4NFm-(1->. This is the same sequence determined by Vinogradov et al. for the F. tularensis O-antigen (Vinogradov E V, Shashkov A S, Knirel Y A, Kochetkov N K, Tochtamysheva N V, et al. (1991) Structure of the O-antigen of Francisella tularensis strain 15. Carbohydr Res 214: 289-297). Assignments for the individual spin systems are shown in Table 8 and FIG. 7 with the GalNAcAN subunits labeled A and B from the non-reducing to the reducing end of the molecule.

TABLE 8
NMR Chemical Shift Assignments
Sugar

| α-D- | H1 | H2 | H3 | H4 | H5 | |
|---|---|---|---|---|---|---|
| GalNAcAN | 5.09 | 4.07 | 4.26 | 4.51 | 4.86 | |
| A (non- | C1 | C2 | C3 | C4 | C5 | |
| reducing) | 100.1 | 53.7 | 73.1 | 82.2 | 72.8 | |
| α-D- | H1 | H2 | H3 | H4 | H5 | |
| GalNAcAN | 5.4 | 4.24 | 4.02 | 4.44 | 4.04 | |
| | C1 | C2 | C3 | C4 | C5 | |
| | 100.9 | 51.8 | 70 | 78 | 70.1 | |
| β-D- | H1 | H2 | H3 | H4 | H5 | H6 |
| QuiNAc | 4.75 | 3.72 | 3.66 | 3.34 | 3.44 | 1.2 |
| | C1 | C2 | C3 | C4 | C5 | C6 |
| | 104.4 | 57.5 | 82.4 | 78.9 | 74.4 | 18.9 |
| β-D- | H1 | H2 | H3 | H4 | H5 | H6 |
| Qui4NFm | 4.41 | 3.53 | 3.52 | 3.62 | 3.43 | 1.19 |
| | C1 | C2 | C3 | C4 | C5 | C6 |
| | 105.3 | 83 | 76.8 | 58.5 | 73.3 | 19.5 |

Cross-glycosidic HMBC crosspeaks were observed between GalNAcAN(A) C1 and GalNAcAN(B) H4 as well as between GalNAcAN(B) C1 and QuiNAc H3, although no inter-ring HMBC crosspeaks were observed involving Qui4NFm. A number of NOEs that were also observed between subunits are summarized in Table 9.

TABLE 9
NOEs Observed Between Sugar Moieties
Inter-ring NOEs observed (grouped by linkage)

| | | |
|---|---|---|
| GalNAcAN(A) H1 | GalNAcAN(B) H4 | glycosidic |
| GalNAcAN(B) H1 | QuiNAc H3 | glycosidic |
| | QuiNAc H4 | |
| | QuiNAc H6 | |
| GalNAcAN(B) H2 | QuiNAc H3 | |
| QuiNAc H1 | Qui4NFm H2 | glycosidic |
| Qui4NFm H1 | GalNAcAN(A) H4 | glycosidic |
| | GalNAcAN(A) H5 | |
| Qui4NFm H2 | GalNAcAN(A) H4 | |

Additional possible NOEs*

| | |
|---|---|
| GalNAcAN(A) H1 | GalNAcAN(B) H3, H5 |
| GalNAcAN(A) H2 | GalNAcAN(B) H4 |
| QuiNAc H1 | Qui4NFm H1, H3 |
| QuiNAcH6 | Qui4NFm H3, H4, and formyl |
| Qui4NFm H1 | GalNAcAN(A) H3 |

*the NOEs listed in this section have characteristics that make them less than ideal, i.e. they fall as a shoulder of another peak, under an intra-ring NOE, are very weak, or lie near the residual water signal.

One result from the NMR data that was unexpected is the presence of a number of stronger resonances in the HMQC spectrum. The total number of these stronger signals, as well as just those in the anomeric region of the spectrum, would seem to indicate the presence of two or three additional sugars or breakdown products of the identified sugars. The MS data previously described, however, is clean and only indicates the presence of a single tetrasaccharide repeat. A more intense resonance in an NMR spectrum can arise in one of two circumstances. The first is that there is a greater concentration of that spin relative to the rest of the spins in the spectrum. This possible explanation could point to the presence of a contaminant, possibly a short oligosaccharide that co-purified with the capsular polysaccharide. The second way more apparently intense peaks can arise in a spectrum is if the spins giving rise to those resonances are more mobile (i.e. faster relaxing) than the rest. This explanation could point to a more flexible substituent branched off the main polysaccharide. Which possible explanation, if either, is correct remains unclear.

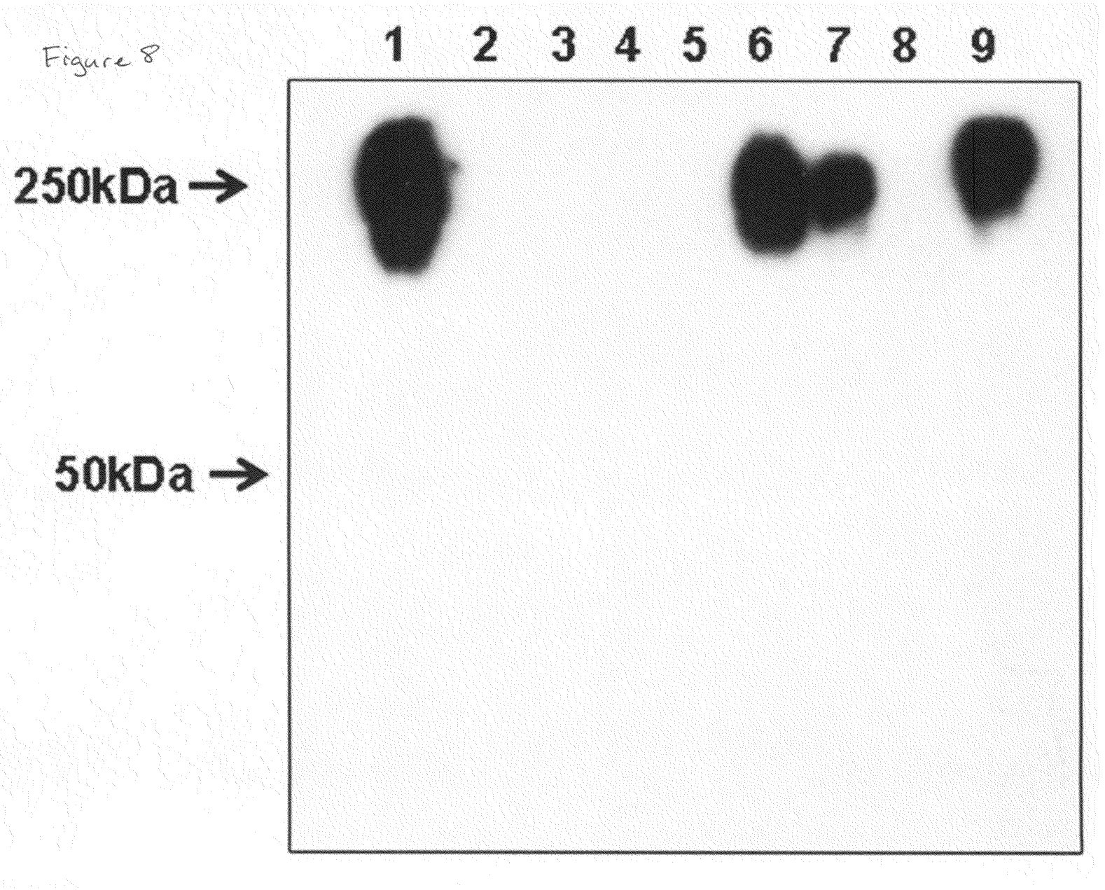

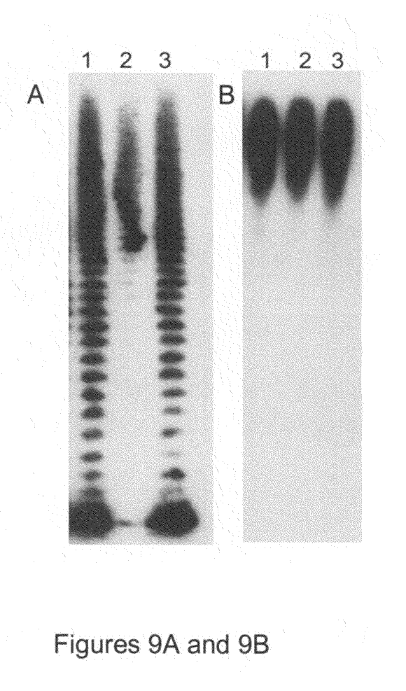

Studies of capsule expression in F. tularensis biosynthesis pathway mutants. Using mutants in F. tularensis LVS of glycosyltransferases and putative capsule biosynthesis genes (cap) obtained from Dr. Dara Frank of the Medical College of Wisconsin, we were able to confirm that the LPS O-antigen biosynthesis pathway plays a role in the biosynthesis of the F. tularensis capsule (Maier T M, Havig A, Casey M, Nano F E, Frank D W, et al. (2004) Construction and characterization of a highly efficient Francisella shuttle plasmid. Appl Environ Microbiol 70: 7511-7519). These studies have demonstrated that mutations that disrupt glycosyltransferase genes involved in O-antigen biosynthesis (wbtI, wbtA1, wbtA2, wbtM, wbtI and wbtC) resulted in the loss of capsule expression (FIG. 8). Mutations in F. tularensis LVS capB and capC had no effect on capsule expression. However, a mutation in wbtK resulted in loss of high mass O-antigen expression but did not affect capsule expression (data not shown). The Kdo-dependent acyltransferase mutant, F. tularensis lpxL, which expresses a portion of the LPS core region but no O-antigen, still produces capsule (FIG. 9A-9B). F. tularensis LVS FTL0706, which has complete homology to the O-antigen polymerase of E novicida, does not express O-antigens yet still expresses the capsule (Table 1, above). In addition, a Tn5 mutation in the promoter region of E tularensis SCHU S4 between open reading frames FTT0673-0674 resulted in a mutant that produced a capsule but the mutant does not express a full length O-antigen. FTT0673c has been annotated as a gene of unknown function and FTT0674 is annotated as a ribose-phosphate pyrophosphokinase. These studies demonstrate that O-antigen and capsule are linked at a number of points in the biosynthetic and assembly pathway but there appear to be several points in transport and assembly that are different.

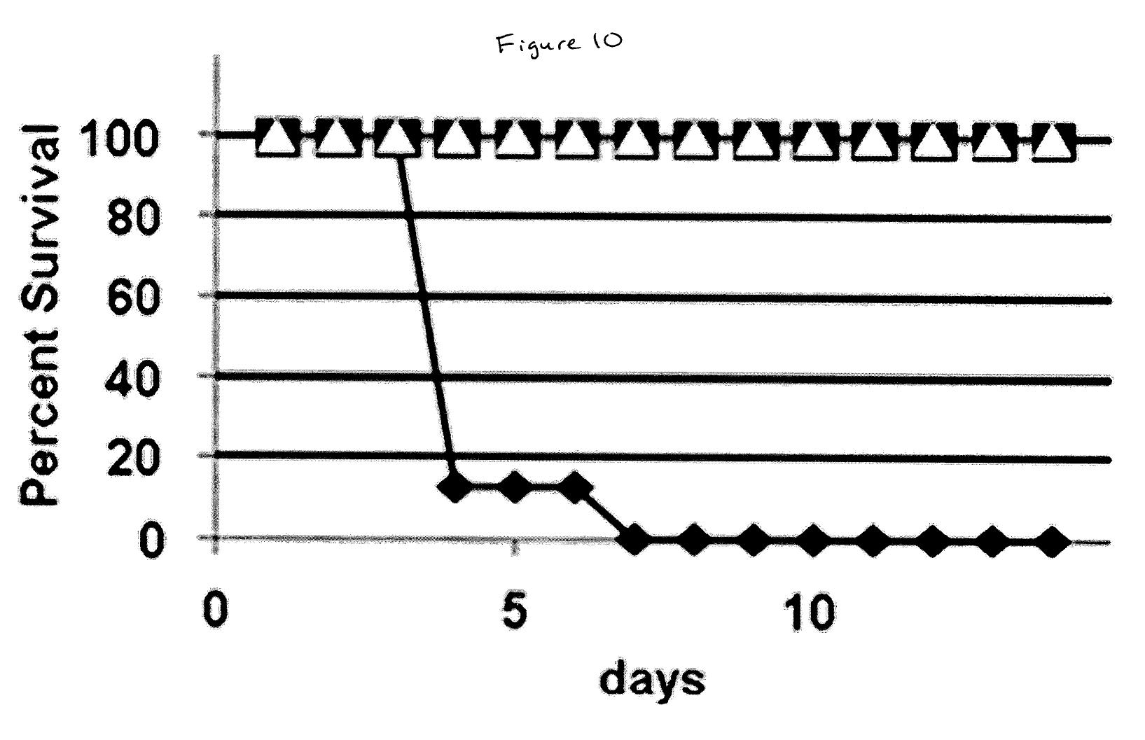

Passive immunization of BALB/c mice with 11B7 MAb against challenge with F. tularensis LVS. We performed an experiment to determine whether mice would be protected from lethal Francisella infection by administration of purified antibody against capsule. Two groups of five mice received 75 μg of purified MAb 11B7 and 24 hours later were challenged intraperitoneally with either 5×10⁴ or 5×10⁵ colony forming units (cfu) of F. tularensis LVS. A third group of mice received 75 μg of a control isotype matched MAb 2C3 that binds to a Neisseria gonorrhoeae membrane protein. All animals were followed for two weeks after challenge. As shown in FIG. 10, administration of MAb 11B7 provided complete protection to BALB/c mice against >100 LD₅₀ doses of F. tularensis LVS for more than 14 days, while all of the mice receiving the control MAb 2C3 succumbed to the infection by day 6. In FIG. 10, survival of all mice given MAb 11B7 was significantly different from the MAb 2C3 control mice (log-rank p=5.1×10⁻⁵), and survival of immunized mice at any one of the two doses of MAb 11B7 was also significantly different from the MAb 2C3 control group (p=1.6×10⁻⁴). The passively immunized mice remained active, did not develop ruffled fur or stop eating or drinking.

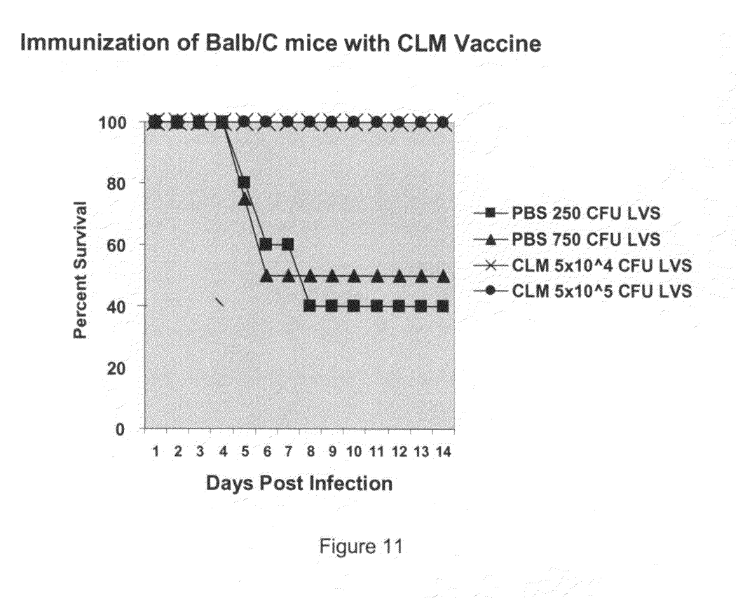

Active immunization of BALB/c mice with capsule and challenge with F. tularensis. To determine if the F. tularensis capsule could protect mice against a lethal challenge, BALB/c mice were immunized intraperitoneally twice 30 days apart with 50 μl containing 10 μg of capsule in PBS mixed with an equal amount of TiterMax Gold adjuvant. Control mice received 50 μl of PBS mixed with the same adjuvant on the same schedule. Eight days after the second injection, a group of five of the control mice was challenged with 250 cfu of F. tularensis LVS. A second group of four control mice received 750 cfu of F. tularensis LVS. One group of five mice immunized with capsule was challenged with 5×10⁴ cfu and a second group of five immunized mice also immunized with capsule received 5×10⁵ cfu of F. tularensis LVS. FIG. 11 shows the results of these studies. These data demonstrated that in the PBS controls samples, 50% of the control mice receiving 250 cfu and 60% of the control mice receiving 750 cfu, died by day 5. All of the mice immunized with capsule survived a challenge of up to 5×10⁵ F. tularensis LVS with no evidence of infection and were euthanized at day 14. In FIG. 11, in spite of the fact that the capsule immunized group of mice received a dose of LVS up to 666 times greater than the PBS control animals, survival of all mice immunized with capsule was significantly different from all the control mice (p=6.9×10⁻³). The actively immunized mice remained active, did not develop ruffled fur or stop eating or drinking. Western blot analyses (FIGS. 12A-12B) and ELISA performed on sera from the immunized mice demonstrated that all had IgG and IgM reactivity to the capsule and no detectable reactivity to F. tularensis LPS. Simultaneous ELISA studies showed that antibody persisted after vaccination for at least 14 days.

### Discussion

Capsular polysaccharides have been shown to be important virulence factors in a wide range of bacterial species. F. tularensis has been considered to be encapsulated but the precise nature of the capsular antigen has never been elucidated. Unreferenced comments in Zinsser Microbiology (Joklik W K (1992) Zinsser Microbiology. Norwalk, Conn.: Appleton & Lange. 1294 p.), state that there is a single immunotype of F. tularensis. Carlisle in 1962 used Ouchterlony plates to compare the immunogenicity of crude extracts from four strains of F. tularensis and a “polysaccharide” isolated from a fifth strain (Carlisle H N, Hinchcliffe V, Saslaw S (1962) Immunodiffusion studies with Pasturella tularensis antigen-rabbit antibody systems. J Immunol 89: 638-644). These studies showed immunological identity between the polysaccharide fraction and the four bacterial extracts, leading to the conclusion that there was a common carbohydrate structure in the five F. tularensis strains. Hood studied a relatively unrefined extract of F. tularensis and did preliminary investigation of sugars isolated from whole bacteria and a “capsular” preparation (Hood A M (1977) Virulence factors of Francisella tularensis. J Hyg (Lond) 79: 47-60). His studies found heptose, glucose, galactose, mannose, rhamnose and possibly dideoxy sugars in the cell wall and mannose, rhamnose and possibly dideoxy sugars in the “capsular” preparation. No further characterization was performed. Studies by Sorokin and Sandstrom have shown that acapsular (based on colony morphology and electron microscopy) F. tularensis strains are sensitive to killing by bactericidal antibody (Sorokin V M, Pavlovich N V, Prozorova L A (1996) Francisella tularensis resistance to bactericidal action of normal human serum. FEMS Immunol Med Microbiol 13: 249-252; Sandstrom G, Lofgren S, Tarnvik A (1988) A capsule-deficient mutant of Francisella tularensis LVS exhibits enhanced sensitivity to killing by serum but diminished sensitivity to killing by polymorphonuclear leukocytes. Infect Immun 56: 1194-1202). Sorokin did not describe how the acapsular status of the strain was produced or verified. Sandstrom used acridine orange mutagenesis to create a mutant library which was screened for “rough” colonies on solid media; the “rough” colonies were designated as lacking capsule. Electron micrographs showed changes consistent with loss of a capsular structure (Sandstrom G, Lofgren S, Tarnvik A (1988) A capsule-deficient mutant of Francisella tularensis LVS exhibits enhanced sensitivity to killing by serum but diminished sensitivity to killing by polymorphonuclear leukocytes. Infect Immun 56: 1194-1202) in the mutants that were selected. However, no studies of the composition or the nature of the mutations leading to the phenotype were described. Using tagged mutagenesis, Su and colleagues recently showed that mutations in an operon, they designated capBCA, led to an avirulent phenoype in mice (Su et al., (2007) Genome-wide identification of Francisella tularensis virulence determinants. Infect Immun 75: 3089-3101). They considered these to be putative capsule genes because of their partial homology to the poly-γ-glutamate capsule biosynthesis locus of Bacillus anthracis. Our studies would suggest that this locus plays no role in the synthesis of the F. tularensis O-antigen capsular polysaccharide (FIG. 8). Whether it encodes for a second capsule-like antigen or another virulence factor is unclear.

We have demonstrated a method for the purification of the F. tularensis capsular polysaccharide and have demonstrated that F. tularensis type A and type B strains produce an O-antigen capsule composed of repeating O-antigen subunits, 4)-α-D-GalNAcAN-(1->4)-α-D-GalNAcAN-(1->3)-β-D-QuiNAc-(1->2)-β-D-Qui4NFm-(1-. Monoclonal antibody FB11, which reacts with the O-antigen, also binds to the capsule. We believe our success in identifying the capsule was due to developing the MAb 11B7 which permitted us to use it as a means of tracking the polysaccharide during isolation since in addition to the O-antigen epitopic structure, this capsule also has an epitopic structure distinct from the LPS as defined by monoclonal antibody 11B7. This antibody was generated by immunizing mice with the capsule preparation which subsequently resulted in the evolution of antibody 11B7. This antibody appears to react only with the capsule suggesting that this is the immunodominant epitope on that structure. It should be noted that in our monoclonal antibody development, using material obtained from saline washed cells as the immunogen, we did not raise an antibody to the O-antigens, suggesting that the LPS was probably in very low amounts or absent from the preparation.

Our studies have shown that the F. tularensis capsule is a repeating polymer of the O-antigen of the LPS. Goldman and co-workers first showed that Escherichia coli 0111 expressed a non-LPS surface polysaccharide composed of O-antigens (Goldman R C, Trus B L, Leive L (1983) Quantitative double-label radiography of two-dimensional protein gels using color negative film and computer analysis. Eur J Biochem 131: 473-480; Goldman R C, White D, Orskov F, Orskov I, Rick P D, et al. (1982) A surface polysaccharide of Escherichia coli 0111 contains O-antigen and inhibits agglutination of cells by O-antiserum. J Bacteriol 151: 1210-1221). Subsequently, a number of Gram-negative bacteria have been shown to express similar structures which consist of large repeating subunits not linked to the LPS core region or lipid A (Nakhamchik A, Wilde C, Rowe-Magnus D A (2007) Identification of a Wzy polymerase required for group IV capsular polysaccharide and lipopolysaccharide biosynthesis in Vibrio vulnificus. Infect Immun 75: 5550-5558; Barak J D, Jahn C E, Gibson D L, Charkowski A O (2007) The role of cellulose and O-antigen capsule in the colonization of plants by Salmonella enterica. Mol Plant Microbe Interact 20: 1083-1091; Chen Y, Bystricky P, Adeyeye J, Panigrahi P, Ali A, et al. (2007) The capsule polysaccharide structure and biogenesis for non-O1 Vibrio cholerae NRT36S: genes are embedded in the LPS region. BMC Microbiol 7: 20; Zhang F, Liu W, Wu X M, Xin Z T, Zhao Q M, et al. (2008) Detection of Francisella tularensis in ticks and identification of their genotypes using multiple-locus variable-number tandem repeat analysis. BMC Microbiol 8: 152).

A review by Whitfield expands the description of the different capsule types and segregates them into specific Groups based on their characteristic structures (Whitfield C (2006) Biosynthesis and assembly of capsular polysaccharides in Escherichia coli. Annu Rev Biochem 75: 39-68). Group 1 and 4 capsules of Enterobacteriaceae are composed of LPS O-antigens (Whitfield C (2006) Biosynthesis and assembly of capsular polysaccharides in Escherichia coli. Annu Rev Biochem 75: 39-68). In strains in which the capsule structure is linked to a lipid A core, it is termed a K_{LPS} to distinguish it from the LPS molecules on the same organism. Other strains may have O-antigen capsules that lack the lipid A core but have another yet unidentified structure which anchors them to the cell wall. These are known as K antigens. Our studies failed to detect lipid A or any of the LPS core sugars as a component of the capsule indicating that the Francisella capsule is probably a K antigen. E. coli group 1 capsules are acidic and typically contain uronic acid structures while group 4 capsules tend to be more diverse and can be distinguished from group 1 capsules by the presence of acetamido sugars in their repeat subunits (Whitfield C (2006) Biosynthesis and assembly of capsular polysaccharides in Escherichia coli. Annu Rev Biochem 75: 39-68). The F. tularensis capsule fits this characteristic by having two 2-acetamido-2,6-dideoxy-O-D-glucose (O-QuiNAc), 4,6-dideoxy-4-formamido-D-glucose (O-Qui4NFm), and 2-acetamido-2-deoxy-O-D-galacturonamide (O-GalNAcAN) in each O-antigen tetrasaccharide subunit.

The genetic organization of capsular genes from a number of bacterial species have been described (Roberts I S (1996) The biochemistry and genetics of capsular polysaccharide production in bacteria. Annu Rev Microbiol 50: 285-315). The assembly and transport systems involved in production of these capsules is complex and include activators of sugar precursors, glycosyltransferases and then a complex of transport and assembly proteins to move these large macromolecules from the periplasm to the cell surface. Group 4 capsule initiation involves transfer of N-acetylglucosamine-1-phosphate to undecaprenol phosphate by WecA followed by the addition of the individual components of the capsular O-antigen subunit to this lipid carrier. The polymerization systems, periplasmic ligase and transport proteins appear to be similar for the group 1 and 4 systems. However, the lengths of the O-antigen repeats in the respective capsules are different. In group 1 the capsule repeats tend to be limited to one or a few O-antigen subunits while the group 4 capsules tend to form long chain O-antigen repeats. The difference in K_{LPS} chain lengths in group 1 and 4 is considered to be due to the absence of wzz (the O-antigen chain length determinant) in isolates with group 1 capsules (Dodgson C, Amor, P., Whitfield, C. (1996) Distribution of the rol Gene Encoding the Regulator of Lipopolysaccharide O-Chain Length in Escherichia coli and Its Infulence on the Expression of Group I Capsular K Antigens. Journal of Bacteriology 178: 1895-1902). In an analysis of the F. tularensis SchuS4 genome using the hidden Markov model, we have homologs of proteins involved in assembly of the capsule, WbtB, Wzx, Wzb and WecA, but no proteins in F. tularensis with homologies to capsular transport proteins in the enteric family, gammaproteobacteria.

Our observation that the F. tularensis lipid A acyltransferase mutant, lpxL and F. tularensis LVS O-antigen polymerase mutant, FTL_{—}0706, did not express repeating O-antigens is not surprising. Analysis of the Western blot using the O-antigen monoclonal antibodies indicated that the polymerase mutant produced an LPS consisting of a core region plus one O-antigen subunit. The fact that the capsule was still expressed in the O-antigen polymerase mutant indicates that the pathways for the polymerization processes are different and suggests that the O-antigen ligase and polymerase are distinct genes in Francisella. The lpxL mutant expresses only a portion of the LPS core region, but still produces a capsule. The reason for the loss of O-antigen expression in the lpxL mutant is less obvious than with the polymerase mutant. Studies in a similar acyltransferase mutant in Neisseria meningitidis serogroup B resulted in an organism which failed to transport LPS to the bacterial surface (Post D M, Ketterer M R, Phillips N J, Gibson B W, Apicella M A (2003) The msbB Mutant of Neisseria meningitidis Strain NMB Has a Defect in Lipooligosaccharide Assembly and Transport to the Outer Membrane. Infect Immun 71: 647-655). It is known that encapsulated Gram-negative bacteria can survive without LPS in the outer membrane (Steeghs L, den Hartog R, den Boer A, Zomer B, Roholl P, et al. (1998) Meningitis bacterium is viable without endotoxin. Nature 392: 449-450). In the N. meningitidis lpxL mutant, evidence of the LPS terminal structures could be identified accumulating in the cytoplasm of the bacterium while no LPS could be identified on the cell surface. This would suggest that in F. tularensis lpxL the O-antigen defect may be related either to inability of the LPS to be transported out of the cell interior or to a failure of the polymerase to add additional O-antigen subunits to the nascent core-O-antigen structure. These results would indicate that the transport pathway and/or assembly pathways for the LPS and capsule are distinct.

Finally, the active and passive immunization studies indicate that the epitope defined by 11B7 is protective against a lethal challenge of F. tularensis LVS in mice. The post-immunization sera indicated that the only epitopic structure detectable was to the capsule in spite of the fact that it is composed of O-antigen subunits. Our IgG and IgM studies in the immunized mice suggested that there was evidence of both isotypes of antibody being generated.

These studies provide evidence for a capsular polysaccharide of F. tularensis. This polysaccharide is a polymer composed of the LPS O-antigen subunit, 2-acetamido-2,6-dideoxy-o-glucose (o-QuiNAc), 4,6-dideoxy-4-formamido-D-glucose (o-Qui4NFm), and 2-acetamido-2-deoxy-o-galacturonamide (o-GalNAcAN). It appears to be conserved across F. tularensis type A and B strains. Passive and active protection assays suggest that this may be a useful immunogen to protect against lethal infection.

### Materials and Methods

Strains and culture conditions: Bacterial strains used in this study are listed in Table 10. All F. tularensis strains were grown at 37° C. on chocolate agar medium supplemented with IsoVitaleX for a final cysteine concentration of 0.1%.

TABLE 10
Strains used in this study

| Strain | Subspecies | source | ref |
|---|---|---|---|
| F. tularensis SCHU S4 | tularensis | BEI | |
| F. tularensis LVS-FDA | holarctica | FDA | A |
| F. tularensis LVS-VT | holarctica | Virginia Tech | B |
| F. tularensis 1547 | holarctica | University | C |
| | | of Iowa | |
| F. tularensis 1547lpxL | holarctica | University | C |
| | | of Iowa | |
| F. tularensis 1547lpxL [plpxL] | holarctica | University | C |
| | | of Iowa | |
| F. tularensis 0673-0674 | tularensis | University | Apicella |
| | | of Iowa | |
| F. tularensis 1623 | holarctica | University | |
| | | of Iowa | |
| F. tularensis T1 0902 | tularensis | Virginia Tech | |
| F. tularensis WY96-3418 | tularensis | BEI | |
| F. tularensis MA00-2987 | tularensis | BEI | |
| F. tularensis NR-50(NIH B-38) | tularensis | BEI | |
| F. tularensis OSPHL 2001- | tularensis | Oregon State | |
| 1011 | | Health Lab | |
| F. tularensis OSPHL 2001-1- | tularensis | Oregon State | |
| 0513 | | Health Lab | |
| F. tularensis OSPHL 2002-1- | not typed | Oregon State | |
| 0990 | | Health Lab | |
| F. tularensis OSPHL 2001-1- | not typed | Oregon State | |
| 0074 | | Health Lab | |
| F. tularensis OSPHL 2001-1- | not typed | Oregon State | |
| 0143 | | Health Lab | |
| F. tularensis LVScapB | holarctica | Medical | D |
| | | College of | |
| | | Wisconsin | |
| F. tularensis LVScapC | holarctica | Medical | D |
| | | College of | |
| | | Wisconsin | |
| F. tularensis LVSwbtC | holarctica | Medical | D |
| | | College of | |
| | | Wisconsin | |
| F. tularensis LVSwbtK | holarctica | Medical | D |
| | | College of | |
| | | Wisconsin | |
| F. tularensis LVS0708 | holarctica | Medical | D |
| | | College of | |
| | | Wisconsin | |
| F. tularensis LVSwbtM | holarctica | Medical | D, E |
| | | College of | |
| | | Wisconsin | |
| F. tularensis LVSwbtK | holarctica | Medical | D |
| | | College of | |
| | | Wisconsin | |
| F. tularensis LVSwbtA1 | holarctica | Medical | D |
| | | College of | |
| | | Wisconsin | |
| F. tularensis LVSwbtA2 | holarctica | Medical | D |
| | | College of | |
| | | Wisconsin | |
| F. tularensis LVS FTT0706 | holarctica | Medical | F |
| | | College of | |
| | | Wisconsin | |
| F. tularensis LVSwbtI | holarctica | Virginia Tech | |
| F. tularensis SCHU S4 0673- | tularensis | University | |
| 0674 | | of Iowa | |
| F. novicida U112 | tularensis | ATCC | |

A = Elkins et al. (2002) In vivo clearance of an intracellular bacterium, Francisella tularensis LVS, is dependent on the p40 subunit of interleukin-12 (IL-12) but not on IL-12 p70. Infect Immun 70: 1936-1948
B = Li et al. (2007) Attenuation and protective efficacy of an O-antigen-deficient mutant of Francisella tularensis LVS. Microbiology 153: 3141-3153
C = McLendon et al. (2007) Identification of LpxL, a late acyltransferase of Francisella tularensis. Infect Immun 75: 5518-5531
D = Maier et al. (2006) In vivo Himar1-based transposon mutagenesis of Francisella tularensis. Appl Environ Microbiol 72: 1878-1885
E = Maier et al. (2007) Identification of Francisella tularensis Himar1-based transposon mutants defective for replication in macrophages. Infect Immun 75: 5376-5389
F = Goddard et al., SPARKY: NMR Assignment and Integration Software. May 2008 ed: University of California, San Francisco

Transposon selection protocol. A Francisella-specific Tn5 transposon system has been developed by our group that is delivered by a temperature sensitive plasmid (Buchan B W, McLendon M K, Jones B D (2008) Identification of differentially regulated Francisella tularensis genes by use of a newly developed Tn5-based transposon delivery system. Appl Environ Microbiol 74: 2637-2645). The plasmid carrying the transposon was purified and introduced into F. tularensis SCHU S4 by cryotransformation (Le Pihive E, Blaha D, Chenavas S, Thibault F, Vidal D, et al. (2009) Description of two new plasmids isolated from Francisella philomiragia strains and construction of shuttle vectors for the study of Francisella tularensis. Plasmid 62: 147-157). Colonies obtained after ˜3 days growth at 30° C. on MMH agar containing 25 μg/ml spectinomycin were inoculated into 5 ml MMH broth with 25 μg/ml spectinomycin and were grown at 30° C. with agitation to an OD₆₀₀ of ˜0.2. Selection of F. tularensis SCHU S4 transposition events was performed at 40° C. with ˜10⁸ organisms plated. Since transposition frequency has previously been shown to be ˜10⁻³, approximately 10⁵ random chromosomal transposon mutants were created by this procedure. Individual F. tularensis Tn5 mutant colonies (˜7,500 mutants) were picked and arrayed into 96-well cell culture plates in 100 μl MMH broth and were incubated at 37° C. until turbid. Freezer stocks were made by adding 100 μl of 2× freezing medium (1.0 M sucrose, 20% glycerol).

Development of hybridomas producing antibodies to the capsule of F. tularensis. BALB/c mice were immunized intraperitoneally with 25 μl of a 1 mg/ml solution of a high salt extract of F. tularensis subsp. tularensis SCHU S4 prepared according to the method of Hood (Hood A M (1977) Virulence factors of Francisella tularensis. J Hyg (Lond) 79: 47-60). Animals were reimmunized at day 21 and day 28. Sera from the immunized mice were tested for antibodies to the immuning extract in ELISA. After confirmation of high antibody titers (>1:10,000), animals were sacrificed 4 days later and their spleens removed. The splenocytes were recovered and fused to SP2/0 murine myeloma cells using 40% polyethylene glycol (1540 MW). Selection was accomplished by plating the fused cells in the presence of hypoxanthine-aminopterin-thymidine DMEM and plated in limiting dilutions to achieve approximately 1-5 cells per well. When colonies appeared, supernatants were tested for presence of antibodies against the saline extract in ELISA. Supernates which proved to be positive in ELISA were tested in Western blot. After this testing, MAb 11B7 fit our criteria as a possible anti-capsular antibody. MAb 11B7 was determined to be an IgG1k using Isostrips (Santa Cruz Biotechnology, Inc, Santa Cruz, Calif.). MAb 11B7 was recloned to assure purity and antibodies produced from these lines for use in this study.

Screening mutants for reduced capsule and identification of the transposon insertion site. In the BSL-3 facility, F. tularensis SCHU S4 Tn5 mutants were inoculated into duplicate 96 well dishes containing MMH broth and were grown until turbid. Cultures were killed by adding 100 μl of 4% paraformaldehyde to each well and incubating overnight. After ensuring sterility, the plates were removed from the BSL-3 facility and the liquid was removed by evaporation. The capsule produced by each organism was then quantitatively determined by ELISA using capsule-specific antibody.

For F. tularensis transposon mutants with reduced capsule production, chromosomal DNA was isolated from individual mutants and digested with EcoRI to create a DNA fragment with the oriR6K origin, the aphA3 gene and flanking chromosomal sequence. The digested DNA was ligated, transformed into a pir⁺ E. coli strain and plated onto agar plates with kanamycin to select for transformants that carried the plasmid of interest. Plasmid DNA was isolated and sequenced using a primer with the sequence 5′-CATGCAAGCTTCAGGGTTGAG-3′ (SEQ ID NO:1) that anneals to the 3′ end of the aphA3 gene and produced sequence of the flanking chromosomal DNA. Sequence data was used to search the sequenced bacterial chromosomal database using NCBI BLAST to identify Tn5 insertion sites within the F. tularensis chromosome.

Isolation of the Francisella capsule. Bacteria were grown as a lawn for approximately 48 h on chocolate agar medium plates at 37° C. and were collected by scraping colonies from the surface and suspending these in a solution of 6 mM Trizma base (Research Products International Corporation, Mt. Prospect, Ill.), 10 mM EDTA (Fisher Scientific, Fair Lawn, N.J.) and 2.0% SDS (w/v) (Research Products International), pH 6.8 containing 50 μg/ml proteinase K and incubated at 65° C. for 1 h and then overnight at 37° C. Additional Tris-SDS solution was added to completely solubilize the bacterial pellet after the 65° C. incubation. The sample was placed at 37° C. in a dry incubator overnight to complete the digestion. To remove the SDS, one tenth the volume of 0.3 M sodium acetate (0.1 ml per 1 ml sample) was added to the samples which were then precipitated with 3 volumes cold 100% ethanol, flash cooled in a dry ice-ethanol bath and incubated overnight at −20° C. Samples were centrifuged for 10 min at 12,000×g at 4° C. and pellets were suspended in deionized water and precipitated a total of three times. Samples were suspended in 15 ml of 10 mM Tris pH 7.4 containing 10 mM CaCl₂ and treated with 80 U micrococcal nuclease (Sigma, St. Louis, Mo.) overnight at 37° C. After incubation, an equal volume of 95% phenol was added to this mixture. Capsule samples and phenol were incubated at 65° C. for 30 min, cooled on ice, and centrifuged at 2,000×g for 10 min at 4° C. The aqueous layer was collected and the phenol layer was back extracted with an equal volume of deionized water pre-warmed to 65° C. The aqueous layers were combined. To remove residual phenol, one-tenth volume of 0.3 M sodium acetate was added to the aqueous layers which were then precipitated three times with 3 volumes of absolute ethanol. The pellet was raised in distilled water and Triton X-114 (Sigma) was added to a final concentration of 5% (v/v). The sample was vortexed for 30 seconds and placed at 4° C. Sixteen hours later, the sample was placed at 37° C. for two hours. The resulting detergent aqueous suspension was centrifuged at 2000×g at 37° C. for ten minutes and the upper aqueous phase removed. The aqueous phase was dialyzed at room temperature against multiple changes of distilled water over 48 h and lyophilized. For some experiments, the lyophilized capsule was reconstituted in 6 mM Tris base, 10 mM EDTA and 2.0% SDS (w/v), pH 6.8 and placed over a 1×20 cm Sephacryl SH500 column equilibrated with the same buffer to remove residual LPS. The void volume of the column was 11 ml and the bed volume was 25 ml. The column was run at 37° C.

Immunological assays. Western blots were performed as previously described using 4-12% gradient gels (Invitrogen, Carlsbad, Calif.) with samples transferred to PVDF (Millipore) (Allen S, Zaleski A, Johnston J W, Gibson B W, Apicella M A (2005) Novel sialic acid transporter of Haemophilus influenzae. Infect Immun 73: 5291-5300). ELISA assays were performed using CoStar 3590 plates (Corning Inc., Corning, N.Y.) coated with 10 μg/ml of capsule as previously described (Allen S, Zaleski A, Johnston J W, Gibson B W, Apicella M A (2005) Novel sialic acid transporter of Haemophilus influenzae. Infect Immun 73: 5291-5300).

Cryoultramicrotomy for Immuno-TEM. F. tularensis colonies were removed intact using a punch and fixed in 4% paraformaldehyde with 0.2% glutaraldehyde in PBS overnight at 4° C. The plug was carefully trimmed into 1 mm square sections, treated with 2.3M sucrose at 4° C. overnight and then frozen by direct immersion in liquid nitrogen. Ultrathin frozen sections (70-95 nm) were cut on Leica EM UC6 Ultramicrotome and transferred onto a carbon-coated formvar covered nickel grid. These samples were then used for immunoelectron microscopy.

Immunogold labeling for electron microscopy. The sections on the nickel grids were washed with PBS and then distilled water. They were blocked with 5% normal goat serum for 15 min. This was followed by incubation with the primary antibody, MAb 11B7 at a 1:100 dilution in PBS for 30 min. The sample was washed with PBS thrice for 2 minutes each time. The secondary antibody was a goat anti-mouse 12 nm gold conjugate diluted 1:40 in PBS and incubated on the sample at room temperature for 30 min. The grid was washed with PBS six times for 2 min each. As a final step, the grids were stained with 0.3% uranyl acetate in 2% methylcellulose for 8 min on ice. The excess solution was removed, the grid dried and the sample viewed with a JEOL 1230 TEM at an accelerating voltage of 120 kv.

Immunogold studies were also preformed on “whole mounted” bacteria with MAb 11B7 to evaluate surface labeling by the antibody directly. To accomplish this, bacteria were suspended in PBS and 5 μl were placed on a formvar coated nickel grid, the bacteria were allowed to settle for 10 minutes and excess fluid blotted off carefully with a Kimwipe. The sample was fixed by flooding the grid with a solution of 4% paraformaldehyde containing 0.05% ruthenium red. Excess fluid was removed with a Kimwipe. Five μl of MAb 11B7 at a dilution of 1:500 was placed on the grid which was incubated at room temperature for 3 hours in a moist chamber. The grid was washed three times in PBS and a goat anti-mouse IgG 12 nm immunogold conjugate was placed on the grid for one hour. The grid was washed three times with PBS followed by distilled water. The grid was dried and viewed with a JEOL 1230 TEM at an accelerating voltage of 120 kv.

Compositional analysis. For composition analyses capsule and LPS samples were run as alditol acetate (AA) and trimethylsilyl (TMS) derivatives. For AA derivatives, approximately 100 μg of sample was hydrolyzed to constituent monosaccharides with 4 N HCl at 100° C. for 6 h. The acid was removed by flushing with dry nitrogen and re-evaporated using a 50% aqueous isopropanol solution. The monosaccharides were re-suspended in water and analyzed on a Dionex ICS-3000 equipped with a CarboPac PA-1 column, using a Pulsed Amperometric Detector (PAD). Samples were eluted using a sodium hydroxide and sodium acetate gradient. A standard set of monosaccharides were analyzed for comparison purposes. Alternatively, the monosaccharides were reduced to the corresponding alditols with either sodium borodeuteride or sodium borohydride overnight at room temperature. Reduced samples were neutralized with a 30% acetic acid solution and excess borates were removed by repeated co-evaporation with acidified methanol and anhydrous methanol several times. Finally, alditols were acetylated using a 1:1 mixture of pyridine:acetic anhydride at 100° C. for 1 h. Reagents were removed using a dry nitrogen flush. Samples were then extracted in dichloromethane and analyzed by GC-MS. For TMS derivatives, either 100 μg (capsule) or 300 μg (LPS) was methanolyzed using 1 M methanolic-HCl at 80° C. for 16 h followed by removal of acidified methanol and re-N-acetylation using CH₃OH:pyridine:acetic anhydride (5:1:1, by vol) at 100° C. for 1 h. Reagents were removed by dry nitrogen flush, and the samples were silylated using Tri-Sil reagent at 80° C. for 30 min. After removal of Tri-Sil by dry nitrogen flush, samples were extracted in hexane and analyzed by GC-MS. Composition analyses of capsule and LPS samples were performed on a Trace-MS Plus GC-MS (Thermo Fisher, Waltham, Mass.) equipped with an AS-2000 autosampler and a Rtx-5 ms column (15 m×0.25 mm, df=0.25 μm) operating in the electron impact (EI) mode. 2 μl of sample was injected into the GC-MS operating in the split mode at 220° C., and the sample was split 1:50. The initial oven temperature was set to 100° C., with a 5 min hold, followed by a ramping of the temperature at 4° C./min, with a final temperature of 270° C. Samples run in the chemical ionization mode (CI) were dissolved in dichloromethane and subsequently analyzed on a Varian GC-MS equipped with a DB-5 column (15 m×0.25 mm, df=0.25 μm [Varian Inc., Palo Alto, Calif.]). 1 μl of sample was injected into the GC-MS, operating with a split ratio of 1:25. The initial oven temperature was set to 120° C., with a 2 min hold, followed by a ramping of the temperature of 5° C./min, with a final temperature of 240° C., with a 5 min hold. Acetonitrile was used as the ionizing gas in CI mode.

Linkage analysis. Linkage analysis of the capsule was done following a modified method of Ciucannu (Ciucannu I (2006) Per-O-methylation reaction for structural analysis of carbohydrates by mass spectrometry. Anal Chim Acta 576: 147-155). First, 0.4 mg of the sample was dissolved in 0.4 mL anhydrous dimethyl sulfoxide (DMSO) overnight, and then a slurry of sodium hydroxide in DMSO was added to the sample and incubated at room temperature for 1.5 h with constant stirring. Then, methyl iodide was added to the sample, followed by a second addition of methyl iodide 30 min later. The permethylated sample was then cooled, and extracted two times with chloroform:water (1:2, v/v). The organic phase was dried and then hydrolyzed at 100° C. for 6 h with 4 N trifluoroacetic acid. The sample was then reduced with sodium borodeuteride and acetylated with pyridine:acetic anhydride (1:1) at 100° C. for 6 h. Samples were then dried under a nitrogen stream, and the partially methylated alditol acetates (PMAA) were dissolved in dichloromethane and analyzed by GC-MS.

MALDI-MS analyses of capsule. Unprocessed capsule was reconstituted in water at a concentration of 3 μg/μl. One μl of the sample was spotted onto a stainless steel MALDI-MS target, allowed to dry, and overlaid with 1 μl of a 50 mg/ml solution of 2,5 dihydroxybenzoic acid (DHB) [Laser Biolabs, Sophia-Antipolis Cedex, France] in 70% acetonitrile. Samples were subsequently analyzed using an LTQ linear ion trap mass spectrometer coupled to a vMALDI ion source (Thermo Fisher). The vMALDI source uses an SI N2 laser (337.3 nm) with a 20-Hz firing rate. Data was collected in the positive ion mode using the automated gain control (AGC) and the automatic spectrum filter (ASF). Tandem mass spectrometry (MSⁿ) data were collected using a precursor ion selection window of 2 m/z and normalized collision energy of 35-40%. Alternatively, samples were analyzed in positive ion mode using the QStarXL equipped with an oMALDI source (Applied Biosystems, Foster City, Calif.).

Mass spectrometric analyses of the capsule for associated lipids. To test for the presence of lipid A in the capsule, various preparations of capsule were generated and analyzed by mass spectrometry. Untreated LPS from F. tularensis strain 1547 was used for comparison purposes. The capsule was treated with hydrofluoric acid (HF) for either 2 hours at 20° C. or 2 days at 4° C. and subsequently dried down using nitrogen gas, under a vacuum, over sodium hydroxide. Samples were further processed by extraction with either CHCl₃:CH₃OH:H₂O (10:5:6) or CHCl₃:CH₃OH (1:1), respectively. The organic phases and precipitate were evaporated to dryness under a nitrogen stream. Untreated samples were reconstituted in water, spotted onto a MALDI target, dried, and overlaid with DHB matrix (50 mg/ml in 70% acetonitrile). Chloroform extracted samples were reconstituted in CHCl₃:CH₃OH (3:1) and overlaid with 6-chloro-3-mercaptobenzothiazole (CMBT) matrix (saturated solution in CHCl₃:CH₃OH (3:1). Samples were analyzed using an LTQ linear ion trap mass spectrometer coupled to a vMALDI ion source (Thermo Fisher) operating in the negative ion mode using the conditions listed above.

NMR methods. An NMR sample was prepared by lyophilizing 4 mg of purified F. tularensis capsule polysaccharide against 3 changes of D₂O and dissolving the dried material in 0.5 mL of 99.96% D₂O from a freshly broken ampoule. All spectra were acquired at 35° C. on a 600 MHz Varian Unitylnova NMR spectrometer equipped with a standard triple resonance probe with pulsed field gradients except for an HMBC experiment which was acquired on an 800 MHz Bruker Avance II NMR spectrometer equipped with a TCI cryoprobe. All spectra were processed using NMRPipe (Delaglio F, Grzesiek S, Vuister G W, Zhu G, Pfeifer J, et al. (1995) NMRPipe: a multidimensional spectral processing system based on UNIX pipes. J Biomol NMR 6: 277-293; Goddard T D, Kneller D G SPARKY: NMR Assignment and Integration Software. May, 2008 ed: University of California, San Francisco) and analyzed using Sparky. The following datasets were used for analysis: ¹³C-HMQC, gHMBC, gCOSY, DQF-COSY, TOCSY (20 ms and 60 ms mixing times), and NOESY (200 ms mixing time). Acquisition and processing parameters are shown in Table 11.

TABLE 11
NMR Spectral Parameters

| | F1 SW | F2 SW | F1 points, | F2 points, | F1 points, | F2 points, |
|---|---|---|---|---|---|---|
| Experiment | (Hz) | (Hz) | acquired | acquired | processed | processed |
| 1H-13C HMQC | 7199.4 | 12062.7 | 512* | 140* | 1024 | 512 |
| DQF-COSY | 5205.6 | 5205.6 | 1024* | 620* | 4096 | 4096 |
| gCOSY | 5205.6 | 2505.6 | 2048* | 1024 | 4096 | 4096 |
| gHMBC | 11029.4 | 38314.2 | 2048* | 700 | 4096 | 2048 |
| TOCSY, 20 ms | 7199.4 | 7199.4 | 1024* | 256* | 4096 | 2048 |
| TOCSY, 60 ms | 7199.4 | 7199.4 | 1024* | 256* | 4096 | 2048 |
| NOESY, 200 ms | 7199.4 | 7199.4 | 512* | 256* | 2048 | 2048 |

Ethics Statements: All animals were handled in strict accordance with good animal practice as defined by the relevant national and/or local animal welfare bodies, and all animal work was approved by the University of Iowa Animal Care and Use committee (ACURF #0808184). Guidelines provided by the NIH were followed in all experimentation. The University of Iowa is PHS assured.

Passive immunization with MAb 11B7 of BALB/c mice and challenge with F. tularensis LVS. BALB/c female mice 6-8 weeks of age were purchased from NCI. Two groups of five mice were injected intraperitoneally with either 50 μg of 11B7 antibody or 50 μg of a matched isotype monoclonal antibody, 2C3, as a negative control. Antibody 2C3 binds to a Neisseria gonorrhoeae membrane protein, H.8. Both MAbs, 11B7 and 2C3, are IgG1 κmonoclonal antibodies. MAb 2C3 was a gift from Dr. Peter Rice, University of Massachusetts. Both antibodies were affinity purified over a Protein G (Thermo Scientific, Rockford, Ill.) column according to manufacturer's instructions. The protein concentration was determined using an Easy Titer Mouse IgG assay Kit (Thermo Scientific, Rockford, Ill.). Twenty four hours post-injection of antibody, both groups of 5 mice were challenged intraperitoneally with 1.2×10⁴ cfu (˜100 LD₅₀) of F. tularensis LVS and the ability of the mice to survive challenge was followed for 14 days. A second experiment was performed using the same protocol for administration of monoclonal antibodies but the challenge dose of F. tularensis LVS was 1.2×10⁵ cfu (˜1000 LD₅₀).

Challenge of BALB/c mice with F. tularensis LVS previously immunized with capsule. BALB/c female mice 6-8 weeks of age were purchased from NCI. Groups of 5 mice were used for each immunization and challenge protocol. Two groups of five mice were injected intraperitoneally with 50 μl of phosphate buffer saline (PBS) and TiterMax Gold adjuvant at a 1:1 ratio. Two separate groups of mice were immunized intraperitoneally with 10 μg of capsule in a 25 μl volume mixed 1:1 with 25 μl TiterMax Gold adjuvant. Mice were bled retro-orbitally 30 days post-immunization to test for the presence of anti-capsule antibodies in the serum. One day post bleeding each group of mice was given a booster dose of antigen and adjuvant identical to the original immunization dose. Two weeks after the boost dose, the mice were bled again and the anti-capsule antibody was re-assessed. Forty eight hours post-bleeding, the groups of mice immunized with PBS, were challenged intraperitoneally with 250 and 750 cfu of F. tularensis LVS. One group of mice, immunized with 10 μg of capsule in adjuvant, was challenged intraperitoneally with 5×10⁴ LVS and the other group of mice immunized with 10 μg of capsule in adjuvant was challenged intraperitoneally with 5×10⁵ cfu of LVS. All mice were followed for 14 days to determine the ability of the mice to survive the challenge.

Statistical Analysis. For the challenge experiments, Kaplan-Meier survival estimates (Kaplan E M, Meier P (1958) Nonparametric estimation from incomplete observations. J Amer Statist Assoc 53: 457-481) of the survival of each group of mice were calculated and differences between groups tested using a log-rank test statistic (Kaplan E M, Meier P (1958) Nonparametric estimation from incomplete observations. J Amer Statist Assoc 53: 457-481; Mantel N (1966) Evaluation of survival data and two new rank order statistics arising in its consideration. Cancer Chemother Rep 50: 163-170). Survival of all control mice were compared to survival of all mice treated with MAb 11B7, and each control group was compared to all immunized mice. All calculations were done in R(R Development Core Team, 2009) and the R survival package (R Development Core TeamR: A Language and Environment for Statistical Computing, 2009. R-Foundation for Statistical Computing, Vienna, Austria. ISBN 3-900051-07-0; Therneau T, Lumley Tsurvival: Survival analysis, including penalized likelihood, 2009. R package version. 2.35-37).

In summary, capsular polysaccharides are important factors in bacterial pathogenesis and have been the target of a number of successful vaccines. Francisella tularensis has been considered to express a capsular antigen but none has been isolated or characterized. We have developed a monoclonal antibody, 11B7, which recognizes the capsular polysaccharide of F. tularensis migrating on Western blot as a diffuse band between 100 kDa and 250 kDa. The capsule stains poorly on SDS-PAGE with silver stain but can be visualized using ProQ Emerald glycoprotein stain. The capsule appears to be highly conserved among strains of F. tularensis as antibody 11B7 bound to the capsule of 14 of 14 F. tularensis type A and B strains on Western blot. The capsular material can be isolated essentially free of LPS, is phenol and proteinase K resistant, ethanol precipitable and does not dissociate in sodium dodecyl sulfate. Immunoelectron microscopy with colloidal gold demonstrates 11B7 circumferentially staining the surface of F. tularensis which is typical of a polysaccharide capsule. Mass spectrometry, compositional analysis and NMR indicate that the capsule is composed of a polymer of the tetrasaccharide repeat, 4)-α-D-GalNAcAN-(1->4)-α-D-GalNAcAN-(1->3)-β-D-QuiNAc-(1->2)-β-D-Qui4NFm-(1-, which is identical to the previously described F. tularensis O-antigen subunit. This indicates that the F. tularensis capsule can be classified as an O-antigen capsular polysaccharide. Our studies indicate that F. tularensis O-antigen glycosyltransferase mutants do not make a capsule. An F. tularensis acyltransferase and an O-antigen polymerase mutant had no evidence of an O-antigen but expressed a capsular antigen. Passive immunization of BALB/c mice with 75 μg of 11B7 protected against a 150 fold lethal challenge of F. tularensis LVS. Active immunization of BALB/c mice with 10 μg of capsule showed a similar level of protection. These studies demonstrate that F. tularensis produces an O-antigen capsule that may be the basis of a future vaccine.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition comprising purified Francisella tularensis polysaccharide capsule (PC).

2. The composition of claim 1, wherein the PC has a molecular weight of 200,000 to 400,000 daltons.

3. The composition of claim 1, wherein the composition contains between 0% and 10% by weight of lipopolysaccharide (LPS) from Francisella tularensis.

4. The composition of claim 1, wherein the composition contains less than 0.1% by weight of lipopolysaccharide (LPS) from Francisella tularensis.

5. The composition of claim 1, wherein the composition contains less than 1% by weight LPS core sugars KDO or mannose.

6. The composition of claim 1, wherein the composition contains less than 1% by weight of lipid A.

7. The composition of claim 1, wherein the PC comprises a tetrasaccharide consisting of one residue of 2-acetamido-2,6-dideoxy-o-glucose (o-QuiNAc), one residue of 4,6-dideoxy-4-formamido-D-glucose (o-Qui4NFm), and two residues of 2-acetamido-2-deoxy-o-galacturonamide (o-GalNAcAN).

8. The composition of claim 1, further comprising a physiologically-acceptable, non-toxic vehicle.

9. The composition of claim 1, further comprising an adjuvant.

10. The composition of claim 1, wherein the PC is operably linked to a conjugation molecule.

11. The composition of claim 10, wherein the conjugation molecule is a peptide, a nucleic acid, or a polysaccharide that is not PC.

12. The composition of claim 10, wherein the conjugation molecule is tetanus toxoid or meningococcal porin.

13. A vaccine comprising an immunogenic amount of purified Francisella tularensis polysaccharide capsule (PC), which amount is effective to inhibit in a patient infection by Francisella tularensis, in combination with a physiologically-acceptable, non-toxic vehicle.

14. A purified antibody that binds specifically to polysaccharide capsule (PC) from Francisella tularensis.

15. The antibody of claim 14, wherein the antibody has the same epitope specificity as hybridoma 11B7, ATCC® Patent Deposit Designation PTA-10595.

16. The antibody of claim 14, wherein the antibody is a human antibody or a humanized antibody.

17. The antibody of claim 16, wherein the antibody is a humanized antibody.

18. The antibody of claim 16, wherein the antibody is a fully humanized antibody.

19. The antibody of claim 16, wherein the antibody is a single-chain Fv or an scFv fragment.

20. A cell of hybridoma 11B7, ATCC® Patent Deposit Designation PTA-10595.
